(19) United States
(12) Patent Application Publication
(10) Pub. No.: US 2024/0401116 A1
(43) Pub. Date: Dec. 5, 2024

(54) NUCLEIC ACID ARRAYS FOR mRNA CHARACTERIZATION

(71) Applicant: INDEVR, INC., Boulder, CO (US)

(72) Inventors: Kathy L. ROWLEN, Boulder, CO (US); Erica Dawson TENENT, Boulder, CO (US)

(73) Assignee: INDEVR, INC., Boulder, CO (US)

(21) Appl. No.: 18/694,860

(22) PCT Filed: Oct. 12, 2022
(86) PCT No.: PCT/US2022/077990

§ 371 Date: Mar. 22, 2024

Related U.S. Application Data
(60) Provisional application No. 63/255,771, filed on Oct. 14, 2021.
(60) Provisional application No. 63/292,747, filed on Dec. 22, 2021.
(60) Provisional application No. 63/339,613, filed on May 9, 2022.
(60) Provisional application No. 63/401,757, filed on Aug. 29, 2022.

(51) Int. Cl. C12Q 1/6837

(52) U.S. Cl. CPC C12Q 1/6837

(57) ABSTRACT

Provided herein are methods and related systems, including assays and kits, for characterization of one or more polynucleotides, including mRNA polynucleotides or other nucleic acid targets. Capture agents are provided on a substrate that are specific to a target region of mRNA in a vaccine or therapeutic sample, wherein the nucleic acid capture agents specifically bind to the target region. Contacting the capture agents with a sample containing relevant mRNA sequences forms a capture agent-target hybridized complex that can be labeled with a variety of detection label agents to generate a measurable signal that may be used for identity, quantification, integrity and/or stability measurements of mRNA in mRNA-based vaccines and therapeutics.

### CROSS-REFERENCE TO RELATED APPLICATIONS

This application claims the benefit of priority to U.S. Pat. App. Nos. 63/255,771 filed Oct. 14, 2021 (Atty Ref. 339036:99-21P US), 63/292,747 filed Dec. 22, 2021 (Atty Ref. 339040:99-21P1 US), 63/339,613 filed May 9, 2022 (Atty Ref. 99-21P2 US), and 63/401,757 filed Aug. 29, 2022 (Atty Ref. 339359:99-21P3 US), each of which are hereby incorporated by reference in their entirety to the extent not inconsistent herewith.

### BACKGROUND

New methods for measuring the identity and quantity of mRNA constructs in mRNA-based vaccines and therapeutics are needed, especially given the 2020 COVID-19 pandemic that precipitated the rapid development of mRNA-based vaccines against COVID-19. Significantly more mRNA-based vaccines are now in development due to the currently licensed mRNA-based COVID-19 vaccines, such as those from Pfizer and Moderna, demonstrating good safety and efficacy. In addition, mRNA shows promise in therapeutics including protein replacement therapy, genome editing, cancer immunotherapies, and others. Analytical methods are needed to measure the integrity and stability of the mRNA constructs to determine whether the mRNA constructs are full-length, and to examine potential degradation as a function of time and storage. New analytical technologies for the characterization of the quality of mRNA-based vaccines and therapeutics that are reagent-sparing, time-saving, and multiplexed will be highly beneficial to the continued development and licensure of new mRNA-based vaccines and therapeutics. Provided herein are methods, devices, and kits related to nucleic acid oligonucleotide microarrays, including to capture and assess identity, quantity, integrity, and stability of mRNA constructs in mRNA-based vaccines and therapeutics.

Typical mRNA constructs included in vaccines include a 5′ cap structure that is essential for translation, protects the RNA from being broken down by blocking exonuclease-mediated degradation, helps the ribosome attach to the RNA to be translated into protein, and prevents innate immune sensing. Also included is a 3′ poly A tail critical for the protection and translation of the RNA molecule. Adjacent to the 5′ cap may be an untranslated region (UTR) that increases stability and maximizes gene expression or translation efficiency, with another UTR adjacent to the 3′ end, again to increase stability and maximize gene expression/translation efficiency. In the center of the construct is a coding sequence or open reading frame that contains the nucleotide sequence that codes for the immunogenic protein produced by the host and is responsible for immunity. Modified nucleotides may also be included to enhance stability and increase the half-life of the resulting mRNA(s).

In addition to the safety and efficacy demonstrated for COVID-19 pandemics for mRNA-based vaccines, one other significant benefit of mRNA vaccines and therapeutics is rapidity of manufacturing. Once a sequence for the protein(s) of interest is/are known, initial batches for pre-clinical and clinical analysis can be generated in just weeks. The manufacturing process is inherently scalable and cell-free and can be adapted to rapidly manufacture any construct. Once the constructs are manufactured and purified, the mRNAs must be combined with a drug delivery system to enable them to reach specific target cells and generate the protein of interest. A common delivery vehicle involves the use of lipid nanoparticles or liposomes that include appropriate cationic or ionizable lipids and other phospholipids that enable the relatively large, fragile, and negatively charged RNA to effectively pass through the cell membrane. The mRNA construct(s) encapsulated within the lipid nanoparticles are then delivered into the body where they are delivered to the cytosol of the host cell, where they are then translated into the protein of interest, which is then presented to the immune system.

To make the mRNA construct(s) of interest, a linearized DNA template is made and then in vitro transcribed in the presence of a DNA-dependent RNA polymerase and the ribonucleoside triphosphates. The residual DNA template is then removed via enzymatic digestion. The mRNA can be capped either during in vitro transcription, or post-transcription through an enzymatic capping reaction. The 3′ polyA tail can be added by inserting the polyA sequence in the starting DNA template, or again via an enzymatic reaction to attach it. Once manufactured, the mRNA(s) must be purified to achieve appropriate purity levels, as the mixture can contain impurities including enzymes, and residual nucleoside triphosphates. In addition to these impurities, the product can also include other nucleic acid-based impurities such as residual DNA template which is subsequently removed with DNase, and a variety of truncated mRNAs or double-stranded RNAs. A variety of chromatographic methods are typically used to purify the mRNA target from these impurities, and these methods can be time-intensive and use toxic reagents. Because the mRNAs are not stable unless enveloped with an appropriate drug delivery system, the mRNAs are then combined into typically a liposome or lipid nanoparticle-based delivery system, although other suitable delivery systems are also in development and in use.

While structural elements as described previously have been included in the mRNA construct to enhance stability, once formulated, the mRNAs are still prone to a variety of degradation pathways during storage that can ultimately affect efficacy of the vaccine. It is critical to be able to monitor integrity of full-length mRNA because a single strand break or oxidation event in the molecule can severely slow or stop translation and therefore have a drastic negative effect on efficacy because the complete protein of interest is not made or is made in much lower quantity. This is in contrast to protein-based vaccines in which a small change in the protein is less likely to have a measurable effect on efficacy, making the stability issue a unique problem in mRNA-based vaccines and therapeutics.

Two main chemical degradation pathways typically occur in vitro including hydrolysis of the phosphodiester backbone (that can be influenced by the mRNA sequence), and oxidation which can lead to base cleavage and strand break. A variety of other degradation mechanisms are possible, including nuclease degradation, enzymatic decapping, and polyA tail shortening, but these are more common in vivo and are minimized through mRNA construct optimization. In combination with the importance of maintaining full-length integrity of the mRNA, these various degradation pathways mean there is a need in the art for reliable and efficient platforms for characterizing mRNA, including for samples under test that may contain one or more unique mRNAs.

This issue has become of extreme importance in view of the rapidly developed COVID-19 mRNA vaccines. Current mRNA assays for identity, quantity, and stability for mRNA vaccines may be sub-optimal, as those were adopted very quickly due to the rapid nature of the vaccine development for addressing the global COVID-19 pandemic. In addition, the rapid development of the mRNA COVID-19 vaccines means that the regulatory guidance regarding recommendations for characterization are still in flux. The currently available assays are often not sequence-specific or take significant time and resources to conduct (both the assay and the downstream data analysis), or both. There is, therefore, a need in the art for a more streamlined assay that meets the necessary requirements. The methods, devices and related kits provided herein address this need.

One method currently used in the manufacturing and quality assessment of mRNA-based vaccines to quantify total mRNA relies on a dye that emits fluorescence once bound to RNA. One commercially available assay is the RiboGreen® assay (sold commercially by Life Technologies/Molecular Probes). The RiboGreen® assay is a 96-well plate-based assay involving a dye whose fluorescence quantum yield is greatly increased upon binding to single stranded RNA compared to free dye in solution. The result is a fluorescence measurement that can be used to quantify mRNA against a standard curve of a reference material. While providing low ng/mL sensitivity, that assay is not sequence-specific, and only provides a measure of total mRNA content, and is not able to distinguish between mRNA that is full-length or fragmented due to degradation.

The RiboGreen® assay is utilized to measure quantity of the naked mRNA in vaccine constructs during upstream manufacturing and is additionally used to determine the percent of mRNA encapsulated in the lipid nanoparticles (LNPs) used as an encapsulation and mRNA delivery vehicle. Once mRNA is encapsulated within the LNPs, the RiboGreen® dye cannot penetrate the LNPs, and therefore is used to measure the free, un-encapsulated mRNA still in solution.

Quantitative reverse transcription-polymerase chain reaction (qRT-PCR) or digital droplet RT-PCR using primers at the start and end of construct can also be used to determine the total amount of transcribable (full-length mRNA). Primer and probe design are likely to be custom for each mRNA construct and are therefore not available off-the-shelf and are typically developed in-house. Also, qRT-PCR can suffer from reliability issues due to the error rate of enzymes used, is not widely used for this application, and cannot be used to determine the different types of degradation that may be occurring.

Ultraviolet (UV) spectroscopy can be used to measure absorbance of the mRNA constructs which can be converted to a concentration using the molar extinction coefficient for the construct in question. Absorbance measurements at 260 nm are used to quantify nucleic acids, with the ratio of absorbance at 260 nm to that at 280 nm used to assess level of protein impurities, and the ratio of absorbance at 260 nm to that at 230 nm used to assess level of organic impurities (with pure RNA typically resulting in a ratio of approximately 2). Those UV-based methods are not sequence-specific and can be sensitive to other impurities in this wavelength range and are generally meant as a qualitative indicator of purity.

Gel electrophoresis can also be utilized to assess mRNA construct molecular weight and integrity using an appropriate molecular weight ladder. It can be used semi-quantitatively with use of an appropriate mass ladder. However, gel electrophoresis methods are typically not off-the-shelf, are labor intensive and time-consuming, and are often developed and optimized in-house and are not available off-the-shelf. In addition, more sophisticated automated capillary gel electrophoresis measurements made with instruments such as the Agilent Bioanalyzer are being employed to separate RNAs by size and to get insight regarding integrity and degradation products. The instrument are consumables are quite expensive, and this methodology cannot typically distinguish between different types of degradation.

Full genetic sequencing of the mRNA construct(s) is often used for identity testing of mRNA. A significant amount of work in the research community has focused on developing specialized RNA sequencing assays that can detect truncated sequences, double-stranded RNA contaminants, RNA molecules with incorrect 5′ termini, unintended internal modifications, and a variety of other target-related impurities. Those are essentially extensions of basic nucleic acid next-generation sequencing methodologies that include additional RNA capture, library preparation, and sequence analysis that enable a more complete picture of species present in RNA mixtures. Drawbacks are that those sequencing assays are relegated to a few academic labs, and data analysis methods are quite sophisticated and non-automated. The library preparation and sequencing can be completed rapidly, typically using Illumina-based methodologies, but all of the protocols are typically developed in-house and are not available as pre-validated, off-the-shelf solutions, and the data analysis can be quite time-consuming. In addition, contract research organizations that provide RNA sequencing are providing consensus sequence services to the greater RNA community, but at the current time are not offering the more sophisticated analyses mentioned above. That analysis is more complicated if extended to a multivalent vaccine containing numerous unique constructs that may share certain sequence features but differ in their coding sequences (otherwise referred to as open reading frames or “ORF”).

A wide variety of chromatographic methods are utilized throughout the manufacturing of mRNA-based vaccines and therapeutics, including reverse phase HPLC, size exclusion HPLC, ion pair reverse phase HPLC, ion exchange HPLC, and others. Those methods can be used to quantify the mRNA against a known standard, and monitor purity and stability. However, methods specifically optimized for mRNA-based vaccines have been relatively slow, and those techniques can be labor intensive and require expensive instruments that need significant upkeep and maintenance, which takes time, money, and personnel resources.

In addition to measuring direct quantity and identity of nucleic acid, the proteins produced by the mRNA construct are typically also measured using an in vitro expression system, either cell-based or cell-free. The translated protein(s) of interest produced are typically assessed via immunoassays or Western blotting or other standard protein assays. While it is critically important to ensure that the mRNAs are making the appropriate protein in vivo and those in vitro assays can be a good indicator of translation efficiency, those protein characterization assays cannot practically substitute for assays that directly identify and quantify the underlying mRNA constructs.

Identity testing of drug substances in vaccines and therapeutics is effectively a quality control test that is required at a number of points in the manufacturing process. Confirming the identity of the drug substance(s) is a required release assay and is particularly important in a facility in which more than one bulk drug substance is being made that gets mixed into a multivalent drug product (such as a multivalent vaccine), or in scenarios where drug substances are shipped between multiple facilities, with each facility performing a piece of the manufacturing process.

In addition to identity, quantification of the drug substance in the bulk drug substances and the final drug product are required for product release. In this case, the quantity of the drug substances is determined generally using a standard for calibration. Preferably, identity and quantity are determined in the same assay, avoiding the need for separate assays.

In addition to determining quantity at the time of manufacture, stability or integrity measurements are also required to ensure the potency of the vaccine or therapeutic are maintained as a function of time in storage. In many cases, these stability or integrity measurements are very similar to quantity measurements. The amount of active or immunogenic drug substance is measured over time to determine stability and ensure minimum potency requirements.

Given the drawbacks of the current methods outlined above, there is a need for new methods for assessing the identity, quantity, and integrity or stability of mRNA in mRNA-based vaccines and therapeutics. In view of the ability to rapidly develop mRNA-based vaccines after a viral genome is sequenced, rapid, efficient and reliable quality control characterization assays are needed to match the rapid vaccine manufacture. This is reflected, for example, by the success of recent mRNA-based vaccines for COVID-19. The unmet needs in rapid multiplexed quantification of mRNA constructs in multivalent vaccine formulations, rapid tracking of mRNA stability, and in the support of bioprocess development, can significantly delay quality control assessments, resulting in attendant delayed approval and roll-out of mRNA-containing products. Manufacturers of these products have a need for alternative methods that can be easily implemented, provide consistency in terms of assay performance between various manufacturing sites, provide multiplexing that enables analysis of all RNAs in a multiplexed product simultaneously, and that has a fast time to result. The time spent developing and optimizing analytical methods for vaccine and therapeutic characterization is a significant resource burden that can be reduced with the use of new methods that provide the aforementioned advantages. Provided herein are methods and related devices or kits for rapid and reliable characterization of any of a number of mRNAs in a product such as a vaccine or therapeutic, in a manner that does not adversely impact bioprocess development. Rather, the methods complement bioprocess development, adding important quality-control assurances that whatever polynucleotide is purported to be in a particular substance, whether an upstream component and/or the downstream end product is, in fact, present, thereby increasing bioprocess development efficiency.

### SUMMARY

The systems and methods provided herein address the above-identified problems in the art by providing a plurality of unique capture agents to a substrate surface, wherein the capture agents hybridize to at least a portion of a polynucleotide in a manner that facilitates characterization of a plurality of characteristics of the polynucleotide. As described herein, selection of capture agents and labels provide the ability to, even in a multiplex manner, assess each polynucleotide's identity, quantity, integrity and stability. This is particularly relevant for polynucleotides that comprise mRNA sequences, such as in an mRNA vaccine or therapeutic, and provides a number of important advantages that align with the nature of the relatively fast roll-out of mRNA vaccines. Advantages include, but are not limited to, rapid and reliable quality control analysis during the mRNA manufacturing bioprocess that is minimally disruptive and can quickly and in the early stages identify issues with the mRNA production process, including specific aspects of the bioprocess.

Provided herein are systems and methods for assessing identity, quantity, integrity and/or stability of polynucleotides, including mRNA polynucleotides. The provided systems and methods may be multiplexed to characterize a multivalent vaccine or therapeutic. Of course, the systems and methods are compatible with singleplex applications. The provided systems and methods may also utilize multiple nucleic acid capture agents specific to different segments of a single mRNA, or mRNAs having different sequences. Reduced testing time and complexity offered by the systems and methods provided herein can reduce costs and allow producers to test vaccine samples during various stages of the production process, allowing for optimization of process steps and rapid assessment of vaccine or therapeutic critical quality attributes. The systems and methods for assessing identity, quantity, and integrity or stability of polynucleotides, is a platform level technology relevant for any number of applications. For example, the technology is applicable for influenza-specific mRNA in a multiplexed influenza vaccine. Of course, the systems and methods provided herein can be used for any mRNA vaccine, including for known viruses and future arising viruses of concern, including arising from new mutations, strains, or newly emergent pathogens. Similarly, the methods and systems are useful for therapeutic applications, where one or more mRNA sequences are provided to a patient in need of a polypeptide or protein encoded by the mRNA. In this manner, also provided herein is a method of using any of the assays provided herein to optimize a bioprocess for making a polynucleotide. Also provided herein is a method of using any of the assays provided herein to assess quality of a product containing one or more mRNA sequences, and any upstream processes thereof.

Accordingly, several advantages with the methods and systems provided herein over conventional mRNA characterization methods include, but are not limited to: rapidly identify mRNA in vaccine bioprocess or therapeutic samples to enhance vaccine and drug development, characterization, or release, to identify one or more mRNAs in a multivalent vaccine using a single test, to quantify one or more mRNAs in a multivalent vaccine using a single test, to measure integrity or stability of mRNAs in a vaccine or drug, to provide a low-cost, simple testing method that a user with minimal technical expertise can perform, and to simplify mRNA identity, quantity, and integrity or stability testing to reduce costs. Other advantages of one or more aspects will be apparent from a consideration of the drawings and ensuing description.

Any of the systems and methods provided herein may be array-based, allowing for multiplexing capability and reducing testing time due to ability to simultaneously assay for multiple targets. The array provided herein is compatible with a range of configurations, including an array on a flat substrate, a bead-based array, or any other type of array commonly known in the art.

The targets of any of the systems and methods provided herein may be comprised of mRNAs, other RNAs, or DNAs, and the targets may include native or non-native ribonucleotides or nucleotides, or a mixture of native and non-native ribonucleotides or nucleotides.

Provided are methods for multiplexed detection of a plurality of targets associated with a monovalent or multivalent mRNA vaccine or therapeutic, the method comprising the steps of: providing a plurality of nucleic acid capture agents that specifically bind to at least a portion of one or more mRNA polynucleotides in a vaccine or therapeutic sample, wherein the nucleic acid capture agents specifically bind to a target region of one or more of the mRNA polynucleotides; contacting the plurality of capture agents with a sample, wherein mRNA polynucleotide(s) in the sample form a hybridized complex with one or more of the nucleic acid capture agents; and detecting a spatial pattern of hybridized complexes.

The method may be for identity testing in a vaccine or therapeutic sample, comprising: 1) providing a substrate with one or more nucleic acid capture agents bound to the substrate, 2) contacting the substrate with a vaccine or therapeutic sample to form a hybridized complex between the nucleic acid capture agent and a polynucleotide, 3) washing away unbound material in the vaccine or therapeutic sample, 4) labeling the complex with one or more label agents to produce a detectable signal, 5) and detecting the presence of polynucleotide(s) in a vaccine or therapeutic sample. Preferably, additional characterization of the polynucleotide(s) is obtained, including one or more of quantity, integrity, or stability.

In one embodiment, a method for identity testing in a vaccine or therapeutic sample from a step in the manufacturing process prior to the mixing of multiple mRNAs and therefore containing a single polynucleotide mRNA produces one or more detectable signals on the array. The signal detected on the array on the one or more nucleic acid capture agents designed to bind the polynucleotide mRNA is positive, as measured by a signal to background ratio exceeding a threshold value such as 3, 5, or 10. The signal(s) detected on the array on the one or more nucleic acid capture agents not designed to bind the polynucleotide mRNA is negative, as measured by a signal to background ratio that does not exceed a threshold value such as 3, 2, or 1.5. The presence of positive signal for the polynucleotide mRNA and absence of positive signal on the nucleic acid capture agents not designed to bind the polynucleotide mRNA (that is, designed to bind other mRNA polynucleotides not present in the sample) may be used to confirm the identity of the target mRNA.

The method may include quantification of one or more targets in a vaccine or therapeutic sample, the targets comprising one or more of mRNAs, other RNAs, or DNAs polynucleotides.

In one embodiment of a method for quantification of one or more polynucleotides in a vaccine or therapeutic sample, one or more calibration curves are constructed using one or more arrays and one or more standardized polynucleotide dilutions at known concentrations. Basically, known but different polynucleotide concentrations are introduced to the arrays and a calibration curve of signal intensity as a function of polynucleotide concentration is obtained. Then, when assaying a sample, the calibration curve is used to quantify the polynucleotide(s) depending on the detected signal. For assays having a plurality of different capture agents specific to a plurality of different polynucleotide sequences, calibration curves are obtained for each of the unique different polynucleotides.

In one embodiment, each different concentration of a serially-diluted standardized target may be contacted with a different array to form bound complexes that are then labeled with an appropriate label agent, imaged with an appropriate imaging system, and the signals may be used to form calibration curves that yield a relationship between quantity of fluorescence signal on the capture agent(s) and initial absolute concentration of the target(s) in the vaccine sample.

In one embodiment, one or more concentration curves may be then utilized to determine the concentration of one or more targets in a sample by contacting an array with a sample containing one or more targets to form complexes between the targets and nucleic acid capture agents, labeling the targets with one or more label agents, and comparing the signals generated from the labeled targets to the calibration curve to back-calculate concentration of the one or more targets in the sample.

In one embodiment, quantification may be performed to assess integrity or stability of a vaccine or therapeutic sample, for example, as a function of time and storage conditions.

In one embodiment, the method and systems of the current invention may be used to determine the identity or quantity of an influenza mRNA in a vaccine sample by contacting an array comprised of a plurality of nucleic acid capture agents, each of which may be complementary to a specific subtype or lineage of influenza virus with a sample containing one or more influenza targets to form complexes between the target and influenza target-specific nucleic acid capture agents, labeling the target with one or more labeling agents, and examining the signals generated on the nucleic acid capture agents to determine the identity or quantity of the target.

In another embodiment, the vaccine sample may be a quadrivalent influenza vaccine sample, and the method and systems allow for simultaneous identification of all four targets in the sample.

In one embodiment, the array of the methods and systems of the current invention is designed such that the nucleic acid capture agents bind a region of the hemagglutinin (HA) or neuraminidase (NA) gene segments that is conserved amongst all of a targeted subtype or lineage so as to enable a method or system of identity testing or quantification of the mRNA components in a seasonal trivalent or quadrivalent influenza vaccine sample without the need to update the nucleic acid capture agents as the vaccine strains change from season to season. By having the capture agent target conserved regions of the mRNA, the need to update the capture agents to accommodate genetic drift year-over-year is avoided. Accordingly, the methods and arrays provided herein may have capture agents selected so that the methods and arrays may be used with influenza vaccines over multiple years without changing the capture agent sequences, such as two or more, three or more, four or more, five or more, or between 3 and 10 years.

In one embodiment, the methods or systems may be used to assess the integrity of an mRNA construct and confirm the presence of each portion of the mRNA construct sequence, such as by designing nucleic acid capture sequences so that each portion of the mRNA construct, including but not limited to elements such as the 5′ cap, the untranslated region, the coding sequence, and the 3′ polyA tail, may be detected to assess the presence of each sequence portion or to quantify each sequence portion. Assessing the presence of each sequence portion may entail interpreting the signal pattern generated after labeling the complexes between the target and the nucleic acid capture agents with appropriate data analysis algorithms such as artificial intelligence-based pattern recognition algorithms.

In one embodiment, the method and systems may be used to determine the identity or quantity of a coronavirus mRNA in a vaccine sample by contacting an array comprised of a plurality of nucleic acid capture agents, each of which may be complementary to a portion of a coronavirus mRNA construct contained in a vaccine sample to form complexes between the target and coronavirus target-specific nucleic acid capture agents, labeling the target with one or more labeling agents, and examining the signals generated on the nucleic acid capture agents to determine the identity or quantity of the mRNA polynucleotides.

In one embodiment, the method and systems may be used to determine the identity or quantity of an mRNA in a therapeutic, such as an immunotherapeutic or other drug or therapeutic by contacting an array comprised of a plurality of nucleic acid capture agents each of which may be complementary to a portion of a therapeutic mRNA construct contained in a sample to form complexes between the target and therapeutic-specific nucleic acid capture agents, labeling the target with one or more labeling agents, and examining the signals generated on the nucleic acid capture agents to determine the identity of the target.

In one embodiment, the method and systems may include contacting an array comprised of a plurality of nucleic acid capture agents with an appropriate mRNA polynucleotide to which has been added at least one component or step capable of linearizing or relaxing secondary or tertiary structure of mRNA to promote effective hybridization.

In one embodiment, the component capable of linearizing mRNA is one or more complementary oligonucleotides added to the mRNA-containing solution such that the one or more complementary oligonucleotides bind to a portion of the mRNA construct that is not overlapping but is adjacent to the mRNA target regions intended to bind to either the capture agent or the label agent (that is, binds within about 5, 4, 3, 2, or 1 nt of the regions intended to bind either the capture agent or the label agent) for the purpose of relaxing or eliminating secondary structure of the mRNA near the capture agent binding region to promote mRNA binding to the microarray.

In another embodiment, the component capable of linearizing mRNA is an additive known to promote relaxation of RNA secondary and tertiary structures such as formamide, urea, Denhardt's solution, dimethyl sulfoxide (DMSO), spermidine, low salt concentration solutions, solutions containing low concentrations of divalent cations such as Mg²⁺, surfactants or detergents.

In another embodiment, the step capable of linearizing mRNA is a heating step, such as at temperatures ranging from 50° C. to 90° C. for times such as 1 minute to 15 minutes. In another embodiment, the heating step is followed by a cooling step so that the temperature is below the Tm above which hybridization cannot reliably occur.

In another embodiment, the vaccine sample may be a monovalent or multivalent coronavirus mRNA vaccine sample. The method and systems allow for simultaneous identification of all targets in the sample.

Provided are arrays or microarrays for performing any of the methods described herein. Provided herein are systems that include arrays or microarrays and other related components, such as optical light sources, filters, detectors and related optical components and computer software and hardware for measuring optical signals from the arrays and characterizing the polynucleotide(s). Also provided are kits comprising the array, labels, controls, and instructions for carrying out any of the methods described herein with the kit.

Provided herein are methods for characterizing a polynucleotide, such as by providing a capture agent on a substrate surface, introducing a sample containing the to-be-characterized polynucleotide to the capture agent, and binding at least a target region of the polynucleotide to the capture agent to form a polynucleotide and capture agent complex. Unbound material is removed from the substrate surface. The polynucleotide and capture agent complex are labeled with a label to form labeled complexes and the labeled complexes detected, including for optical-based labels by an optical detector to thereby characterize the polynucleotide.

The method may be for multiplex characterization of a plurality of mRNAs, including by using a plurality of capture agent populations, with each capture agent population specific to a unique polynucleotide, and more specifically to a unique target sequence.

Depending on the application of interest and desired characterization, the characterizing may comprise one or more of: polynucleotide identity; polynucleotide quantity; polynucleotide integrity; and/or polynucleotide stability.

The sample may be from an mRNA vaccine. For example, it may be desired to characterize one or more constituents of the mRNA vaccine, such as one or more polynucleotides that form the mRNA vaccine.

The polynucleotide may comprise one or more of: a native ribonucleotide; a native nucleotide; a non-native ribonucleotide; and/or a non-native nucleotide. For example, the polynucleotide may be DNA based and/or RNA based.

The methods provided herein are particularly suited for multiplex characterization of a plurality of polynucleotides. For example, the method may comprise providing a plurality of unique capture agents on the substrate surface to provide multiplex characterization of a plurality of polynucleotides. The multiplex characterization may be used to: identify a plurality of mRNAs in a vaccine manufacture process; identify a plurality of mRNAs in a therapeutic; characterize a plurality of mRNAs in a vaccine; and/or characterize a plurality of mRNAs in a therapeutic. The multiplex configuration is particularly useful for improving efficiency of mRNA characterization in an application having a plurality of mRNA polynucleotides. Instead of running a plurality of assays for each of the unique mRNA polynucleotides, one sample can be provided to one substrate having a plurality of unique capture agents.

The capture agent may comprise a plurality of unique capture agents configured to specifically hybridize to a plurality of unique target polynucleotide sequences, wherein each individual unique capture agent specifically hybridizes to one unique target polynucleotide sequence, thereby providing multiplex detection.

The plurality of capture agents can be provided on the substrate surface in an array, and the detecting step may comprise detecting a spatial pattern of complexes. The detecting may be by optical detection.

The detecting step may comprise assigning a threshold value relative to an optical signal generated by the labeled complexes and identifying a positive complex at locations on the substrate surface for the optical signal generated by the labeled complex that exceeds the threshold value. The identifying may be automated by an automated optical detector that measures optical intensity of the optical signal. For regions where the intensity is less than the threshold value, a non-binding (e.g., negative complex) is identified. Similarly, for regions having an intensity greater than or equal to the threshold value, a complex is identified. The method may further comprise the steps of identifying a negative complex at locations on the substrate surface for an optical signal that is less than the threshold value; and using the combination of positive complex locations and negative complex locations to thereby characterize the polynucleotide. In this manner, the method may automatically characterize the polynucleotide, including each polynucleotide in a multiplex sample having a plurality of unique polynucleotide target sequences.

The method may further comprise the steps of introducing a standardized polynucleotide having a known polynucleotide concentration to the substrate surface; binding the standardized polynucleotide to the capture agent to form a standardized polynucleotide and capture agent complex; removing unbound material from the substrate surface; labeling the standardized polynucleotide and capture agent complex with the label to form labeled standard complexes; and detecting the labeled standard complexes. The previous steps are repeated for the standardized polynucleotide having a different known polynucleotide concentration and a calibration curve is generated from the plurality of standardized complexes. In this manner, the various different known polynucleotide concentrations are selected to span the range of expected concentrations for the application of interest to ensure the detection is within the calibration range. The calibration curve is used to quantify an amount of the polynucleotide in the sample. Preferably, at least three, four, five, or between three and sixteen unique standardized polynucleotides, each having a different unique polynucleotide concentration, are used to generate the calibration curve.

The method is compatible with various detection modalities. For example, the method and related devices may configure the capture, the label, or both, to provide target specificity.

The capture agent may comprise a plurality of unique capture agents provided in an array on the substrate surface, with each unique capture agent specific to a unique target polynucleotide sequence and the label is a universal label. In this aspect, the capture sequences are unique, but the label is universal (for example, to the 3′ polyA tail or the 5′ cap which is conserved over all the polynucleotide targets).

Alternatively, the capture agent may comprise a universal capture agent configured to bind to a conserved region of a plurality of unique target polynucleotide sequences, and a plurality of labels, each label configured to bind to a unique complex. In this aspect, the capture agent is universal (e.g., to the 3′ polyA tail or the 5′ cap), but the label targets a unique portion of each polynucleotide target.

The capture agent may comprise a plurality of capture agents and a plurality of labels, each of the plurality of capture agents configured to bind to the unique target polynucleotide and each of the plurality of labels configured to bind a unique target region of the polynucleotide sequences. In this aspect, both the capture agent and the label agent are unique for each of the polynucleotide targets.

The characterization may comprise assessment of integrity or stability of the polynucleotide as a function of time and/or storage conditions.

The sample may be an mRNA-containing vaccine or therapeutic against a virus of interest or a genetic disease. The sample may be a virus of interest, such as a coronavirus, an influenza virus, and/or an emergent virus. The sample may be an mRNA influenza vaccine, and the capture agent has sequence complementarity to a specific subtype or lineage of influenza virus. The capture agent may comprise a plurality of unique capture agents, each capture agent having sequence complementarity to a different subtype or lineage or strain of influenza virus.

The detecting step may further comprise determining an identity and/or quantity of the polynucleotide that is mRNA in the sample.

The capture agent may comprise a first capture agent configured to specifically hybridize to an mRNA corresponding to a conserved region of influenza hemagglutinin (HA) gene; and/or a second capture agent configured to specifically hybridize to an mRNA corresponding to a conserved region of influenza neuraminidase (NA) gene; wherein the conserved HA and NA regions are conserved amongst all of a targeted influenza subtype or lineage so that the method of characterizing the mRNA can characterize different seasonal influenza mRNA vaccines without updating the capture agents. This is particularly beneficial in that the need to change out assays for different seasons is avoided, without adversely impacting assay reliability and sensitivity.

The sample may be a multivalent mRNA vaccine, including a quadrivalent mRNA influenza vaccine.

The characterizing may correspond to confirming a presence of a portion of an mRNA construct sequence, including the portion of mRNA corresponding to: a 5′ cap, an untranslated region, a coding sequence, or a 3′ polyA tail.

The sample may comprise a therapeutic having mRNA for a genetic disease treatment.

The method may further comprise the step of linearizing a target polynucleotide sequence of the polynucleotide to facilitate target polynucleotide sequence binding to a respective capture agent by relaxing or eliminating polynucleotide secondary structure. The linearizing may comprise introducing to the sample one or more of: heat, a chaperone, an additive and/or a detergent.

The method may be used in a quality control application. For example, the method may be incorporated into a manufacturing process that makes one or more components of a vaccine or therapeutic containing mRNA. Use of the method at different process time-points can efficiently identify quality control problems, including related to mRNA sequence integrity, so that the process may be stopped and issues addressed, thereby minimizing impact on the overall manufacturing process.

The quality control application may be a multivalent mRNA vaccine, and one configuration of capture agents on the substrate surface is used for all quality control assessments related to singleplex characterization of mRNA vaccine individual polynucleotide constituents and multiplex characterization of all mRNA polynucleotides in the multivalent vaccine.

Also provided herein are devices, such as a microarray mRNA characterization system configured to perform any of the methods provided herein, including to characterize one or more mRNA polynucleotides.

### BRIEF DESCRIPTION OF THE DRAWINGS

Non-limiting and non-exhaustive embodiments of the technology of the present application, including the preferred embodiment, are described with reference to the following figures, wherein like reference numerals refer to like parts throughout the various views unless otherwise specified.

FIG. 1 illustrates one embodiment of a microarray on a substrate that is a flat surface, such as a slide. The microarray can comprise replicate arrays.

FIG. 2A-2B is a schematic illustration of the capture agent-target mRNA complex. The target mRNA may bind in either the 5′ to 3′ or the 3′ to 5′ direction, depending on the relevant sequence in the capture agent (compare FIG. 2A and FIG. 2B). The capture agent can be a polynucleotide capture agent immobilized on a substrate or array surface. To facilitate production of a readable signal, the complex may be labeled with an oligonucleotide label.

FIGS. 3A-3B is a schematic illustration of a singleplex application. FIG. 3A illustrates a labeled complex from a sample comprising a single mRNA target, although multiple polynucleotide capture agents are provided on the substrate for detecting other mRNA targets. In this example, the array is compatible with any samples that may contain one of multiple potential target mRNAs (such as in a bioprocess sample containing one component of a multivalent vaccine or therapeutic). Relevant information is obtained by detection of the label having complementary oligonucleotide sequence to the target mRNA and by the absence of capture of target mRNA on alternative polynucleotide capture agents that are non-complementary to the target mRNA. FIG. 3B is a schematic illustration of the most “simple” form of an array for singleplex applications, with only one single capture agent provided on the substrate to detect only one mRNA target, useful for characterizing samples where only one mRNA target is of interest (such as in a sample from a monovalent vaccine or therapeutic). Alternatively, this detection scheme may be utilized for characterizing total mRNA without discriminating characterizing each mRNA target independently in a sample containing multiple mRNA targets.

FIG. 4 is a schematic illustration of a plurality (e.g., four) of unique complexes. The capture and fluorescence labeling of four unique target mRNAs by polynucleotide capture agents and labels each with complementary oligonucleotide sequences to their respective target mRNA reflect the platforms provided herein are compatible with multiplexed characterization of a plurality of mRNA targets.

FIG. 5 is a schematic illustration of the capture of a single target mRNA by an polynucleotide capture agent with a sequence complementary to the target mRNA and with dual labels. The target mRNA is subsequently labeled with a first label having a sequence complementary to the target mRNA and having a fluorescent molecule with first emission wavelength capable of being detected by a first detection channel, and with a second label having a sequence complementary to a different portion of the target mRNA and a fluorescent molecule with a second emission wavelength capable of being detected by a second detection channel, wherein the first and second emission wavelengths are different.

In this manner, the platform is capable of utilizing two color detection to characterize the complex as corresponding to intact mRNA. Full length characterization is provided by ensuring labels bind to the 3′ and 5′ ends of the mRNA target. Any degradation of mRNA impacts label binding, and therefore decreases a detectable fluorescence signal.

FIG. 6 schematically illustrates another multiplex assay. A plurality of unique (e.g., four) oligonucleotide capture agents bind multiple unique target mRNAs to form a plurality of unique complexes. The target mRNAs are subsequently labeled with a first set of labels each having a sequence complementary to one of the unique target mRNAs and each having a fluorescent molecule with a first emission wavelength capable of being detected on a first detection channel, and with a second set of labels each having a sequence complementary to a different portion of each of the unique target mRNAs and a fluorescent molecule with a second emission wavelength capable of being detected by a second detection channel, wherein the first and second emission wavelengths are different. In this manner, the platform is capable of utilizing two color detection to characterize the complex as corresponding to intact mRNA. In this manner, the assay is similar to FIG. 5, but is capable of characterizing a plurality of unique intact mRNAs. The characterization is exemplified as both identity and integrity of each of a plurality of mRNAs.

FIG. 7 is a flow-chart summary for making a multivalent vaccine illustrating the various process points the instant assays may be utilized during and after vaccine production.

FIG. 8 is a schematic illustration of a capture agent-target-label agent mRNA complex that facilitates the measurement of full-length, intact mRNA construct. In this embodiment, the target mRNA is binding to a polyT polynucleotide capture agent immobilized on the microarray via the 3′ polyA tail of the target mRNA. The target mRNA is subsequently labeled with a fluorescently-conjugated anti-5′ cap antibody to form a capture agent-target-label agent complex that can be detected via fluorescence imaging.

FIG. 9 is a schematic illustration of the capture agent-target-label agent mRNA complex that facilitates the measurement of full-length, intact mRNA construct. In this embodiment, the target mRNA is binding to a polyT polynucleotide capture agent immobilized on the microarray via the 3′ polyA tail of the target mRNA. The target mRNA is subsequently labeled with a fluorescently-conjugated polynucleotide capture agent that is complementary to a sequence region on the target mRNA adjacent to its 5′ end to form a capture agent-target-label agent complex that can be detected via fluorescence imaging.

FIG. 10 illustrates a representative microarray layout (top left panel) that comprises a poly T capture agent, a resultant fluorescence image (top right panel) after the hybridization of target GFP mRNA comprising a polyA tail and a GFP coding region capable of being captured by the poly T capture agent, and example data (bottom panel graph) demonstrating a dose dependent response curve on the poly T capture agent for a serial dilution of GFP mRNA on the microarray.

FIG. 11 is a schematic illustration of a capture agent-target-label agent mRNA complex that facilitates measurement of a specific target mRNA. In this embodiment, the target mRNA is binding to a polynucleotide capture agent immobilized on the microarray that is complementary to a portion of the coding region of the target mRNA. The target mRNA is subsequently labeled with a fluorescently-conjugated anti-5′ cap antibody to form a capture agent-target-label agent complex that can be detected via fluorescence imaging.

FIG. 12 is a schematic illustration of a capture agent-target-label agent mRNA complex that facilitates measurement of a specific target mRNA. In this embodiment, the target mRNA is binding to a polynucleotide capture agent immobilized on the microarray that is complementary to a portion of the coding region of the target mRNA. The target mRNA is subsequently labeled with a fluorescently-conjugated polynucleotide capture agent complementary to a sequence region on the target mRNA adjacent to its 5′ end to form a capture agent-target-label agent complex that can be detected via fluorescence imaging.

FIG. 13 is a schematic illustration of a capture agent-target-label agent mRNA complex that facilitates the measurement of a specific target mRNA. In this embodiment, the target mRNA is binding to a polynucleotide capture agent immobilized on the microarray that is complementary to a portion of the coding region of the target mRNA. The target mRNA is subsequently labeled with a fluorescently conjugated Poly T polynuleotide label agent that binds to the poly A tail of the target mRNA.

FIG. 14 illustrates a representative microarray layout (top left panel) that comprises a polynucleotide capture agent complementary to a portion of the GFP mRNA coding region, a resultant fluorescence image (top right panel) after the hybridization of target GFP mRNA comprising a polyA tail and a GFP coding region capable of being captured by the coding region capture agent and labeled by an anti-5′ cap Ab detection label, and example data (bottom panel graph) demonstrating a dose dependent response curve on the coding region capture agent for a serial dilution of GFP mRNA on the microarray.

FIG. 15 illustrates a representative microarray layout (top panel) that comprises a polynucleotide capture agent complementary to a portion of the green fluorescent protein (GFP) mRNA coding region and a polynucleotide capture agent complementary to a portion of the firefly luciferase (Fluc) mRNA coding region. An example of resulting fluorescence images after the hybridization of target GFP mRNA coding region and FLuc mRNA coding region being captured by the coding region capture agent, and subsequently labeled with anti-5′ cap Ab or anti-Poly T oligo (compare bottom left and bottom right images).

FIG. 16 illustrates exemplary data demonstrating dose-dependent response curves after the hybridization of target monovalent GFP mRNA, monovalent FLuc mRNA, and bivalent GFP/FLuc mRNA. Target mRNA is captured with either a GFP (top panel) or FLuc (bottom panel) mRNA coding region capture agent, and subsequently labeled with anti-5′ cap Ab.

FIG. 17 illustrates exemplary data demonstrating dose-dependent response curves after the hybridization of target monovalent GFP mRNA, monovalent FLuc mRNA, and bivalent GFP/FLuc mRNA. Target mRNA is captured with either a GFP (top panel) or FLuc (bottom panel) mRNA coding region capture agent, and subsequently labeled with a fluorescently conjugated Poly T polynucleotide label agent.

FIG. 18 illustrates schematically that total mRNA in a vaccine sample is the sum of mRNA encapsulated within a lipid nanoparticle (LNP) and remaining free mRNA that is not encapsulated within a LNP.

FIG. 19 shows a graph of a serial dilution of naked mRNA used as a standard along with measurements of mRNA encapsulated in each of two different lipid nanoparticle systems (Pfizer and Precision Nanosystems), and the measured accuracy of the encapsulated mRNA compared to the expected encapsulated mRNA concentration of 3.1 g/mL based on RiboGreen assay measurements.

FIG. 20 shows a schematic illustration of two mRNA constructs, one coding for influenza NA and the other influenza HA, along with the designed capture agents. The center panel shows a microarray layout and representative fluorescence images for monovalent and bivalent analyses of the influenza NA and HA constructs, and the bottom panel highlights specificity of the capture agents for their intended targets for a variety of detection labeling schemes.

FIG. 21 shows linearity with response for monovalent naked or unencapsulated mRNA, with NA mRNA in the left column and HA in the right column for a variety of polynucleotide capture agent and polynucleotide or antibody detection labeling schemes.

FIG. 22 shows similarity of response for monovalent (filled circles) and bivalent (open triangles) 8-point dilution series using polyT polynucleotide detection label for NA mRNA using (a) NA(i) coding region capture agent, (b) NA(iv) coding region capture agent, and for HA mRNA using (c) HA(i) coding region capture agent, and (d) HA(iv) coding region capture agent.

FIG. 23 shows limits of quantification and dynamic ranges for the detection of bivalent influenza NA and HA mRNAs for 2 different polynucleotide capture agents for each mRNA target.

FIG. 24 shows the results of a 3-operator accuracy and precision investigation for bivalent NA and HA mRNA samples for 2 different capture agents for each mRNA target.

FIG. 25A-25B shows representative fluorescence images of naked NA mRNA and LNP-encapsulated mRNA (FIG. 25A) as well as a table of signal to background ratios highlighting specificity achieved in LNP-encapsulated NA and HA mRNA samples (FIG. 25B).

FIG. 26 shows comparative signal responses for monovalent (filled circles) and bivalent (open triangles) LNP-encapsulated NA mRNA in top panels and HA mRNA in bottom panels, indicating similarity of response.

FIG. 27 shows similarity of response for bivalent LNP-encapsulated mRNA (open triangles) and naked mRNA (closed circles) 8-point dilution series using polyT polynucleotide detection label for NA mRNA using (a) NA(i) coding region capture agent, (b) NA(iv) coding region capture agent, and for HA mRNA using (c) HA(i) coding region capture agent, and (d) HA(iv) coding region capture agent.

FIG. 28 shows accuracy and precision for monovalent NA and HA mRNA constructs in LNP-encapsulated samples using naked as a standard on 2 different polynucleotide capture agents for each mRNA.

### DETAILED DESCRIPTION

The technology of the present application will now be described more fully below with reference to the accompanying figures, which form a part hereof and show, by way of illustration, specific exemplary embodiments. These embodiments are disclosed in sufficient detail to enable those skilled in the art to practice the technology of the present application. However, embodiments disclosed herein may be implemented in many different forms and should not be construed as being limited to the embodiments set forth herein. The following detailed description is therefore, not to be taken in a limiting sense. Moreover, the technology of the present application will be described with relation to exemplary embodiments. The word “exemplary” is used herein to mean “serving as an example, instance, or illustration.” Any embodiment described herein as exemplary is not necessarily to be construed as preferred or advantageous over other embodiments. Additionally, unless specifically identified otherwise, all embodiments described herein should be considered exemplary.

As used herein, the term “polynucleotide” refers to a combination of nucleotide monomers that are connected to one another through covalent bonds. A polynucleotide may be single-stranded, and may comprise native DNA bases A, C, T, and G, and may comprise native RNA bases A, C, U, and G. Alternatively, a polynucleotide may comprise non-native bases, or a mixture of native and non-native bases, or other modifications including those resulting in locked nucleic acids or peptide nucleic acids and include oligonucleotides that are manufactured synthetically by methods including, but not limited to, solid-phase chemical synthesis. The polynucleotide may comprise any one or more of mRNA, non-replicating mRNA, self-amplifying mRNA, circular RNA, other RNA, DNA, or other polynucleotide, nucleic acid, or ribonucleic acid molecule present in a vaccine or therapeutic.

The systems and methods provided herein are compatible with any of a range of polynucleotides, depending on the application of interest. For example, in a vaccine context, the polynucleotide may comprise a plurality of mRNAs used in a multivalent vaccine. The specific mRNA sequence will depend on the virus against which the vaccine targets. For influenza vaccines, the polynucleotides will correspond to mRNA from an influenza virus. For COVID-19 vaccines, the polynucleotides will correspond to mRNA from COVID-19 virus. As discussed below, the polynucleotide sequence(s) is important because they inform selection and design of capture agent(s), with the capture agent having at least a portion that is complementary to at least a portion (“target region”) of the polynucleotide sequence.

The term “array” refers to a substrate onto which one or more capture agents are immobilized that are capable of being individually assessed for binding one or more target analytes. The target analytes are generally referred herein as polynucleotide(s). The substrate may be a flat or a curved surface. A slide is one example of a flat surface. A bead is an example of a curved surface. The systems and methods provided herein are compatible with any of a range of substrates known in the art. Array also reflects that the unique capture agent spatial arrangement on a surface is known. In this manner, a spatially-varying optical signal is readily identified with the corresponding spatially-arranged unique capture agents. In this manner, obtaining a spatial map of optical signals over the substrate provides the ability for multiplex characterization of a plurality of polynucleotides in a single array.

As used herein, the term “capture agent” refers to a polynucleotide sequence (e.g., “polynucleotide capture agent”) or antibody, aptamer, or other appropriate molecule immobilized on an array substrate capable of specifically binding or specifically hybridizing to a nucleic acid target such as an mRNA target region. In this context, “target” or “target region” refers to at least a portion of the polynucleotide that is selected to specifically bind to the capture agent. The target region may have a sequence length ranging from between 10-80 nucleotides, so that the capture agent will similarly have a corresponding 10-80 nucleotide length associated with the complementary binding event, including about 20-25 nucleotides. Similar lengths may be provided to the label acting functionally as a probe that hybridizes to its respective label target region on the polynucleotide that may be different than the capture agent target region sequence on the polynucleotide. As described herein, the 3′ and 5′ ends of the polynucleotide sequence may correspond to regions that do not specifically bind to the capture agent, so that a polynucleotide of interest can be generally characterized as having a target region configured to bind to a capture agent and end regions that are not bound to the capture agent. As described, those ends may be configured depending on the application of interest (enhanced packaging, delivery, stability, etc.), including being resistant to degradation (such as by RNases) and/or to provide accessibility for label binding (3′ polyA tail, 5′ cap). Of course, larger lengths of the polynucleotide (longer target regions) may be used to bind to the capture agent, including substantially the entire length, so long as the ability to reliably label is maintained for complex detection.

The term “binding” refers to a sequence-specific attractive interaction between two molecules, such as a hybridization between two complementary polynucleotides, or the association between an antibody or an aptamer and another analyte such as a polynucleotide, protein, or antigen, to form a complex. Complex formation and stability depend on substantial complementarity between the two strands, and, as noted above, a certain degree of mismatch can be tolerated. “Complementary polynucleotides” or “complementary sequence” means a sequence of nucleotides that forms a hydrogen-bonded duplex with another sequence of nucleotides according to Watson-Crick base-pairing rules. For example, the complementary base sequence for 5′-AAGGCT-3′ is 3′-TTCCGA-5′. The term “substantially complementary” refers to a sequence having sufficient complementarity such that reliable characterization, such as arising from complex formation, that the method is reliable. The method and systems provided herein can accommodate less than 100% sequence complementarity. For example, depending on hybridization conditions related to temperature, pH, and the like, as well as sequence length, specificity and the like, the sequences may tolerate some sequence mismatch. For example, the sequences may be greater than 90%, greater than 95%, greater than 98%, or 100% complementarity. For example, for a 20-nucleotide length sequence, the mismatch in sequence complementarity may be two or less, one or less, or no nucleotide complementarity mismatch. Preferably, there is exact complementarity in a middle region of the capture agent, with mismatch tolerance toward the 3′ and 5′ ends. Preferably, sequence complementarity (and sequence length) is selected such that less than 10%, less than 1%, or less than 0.1% of binding is to sequences other than the target polynucleotide.

The potential binding to form a complex may be conducted under hybridization conditions and held at an appropriate temperature until binding via annealing occurs. Thereafter, the array is washed free of extraneous materials, leaving the complexes that can be typically optically detected and quantified detecting an optical pattern. As is well known in the art, if the capture agent and target hybridize by forming a strong non-covalent bond between the two molecules, it can be reasonably assumed that the two molecules are substantially complementary, or completely complementary if the annealing and washing steps are carried out under conditions of high stringency.

The more stringent the conditions, the greater the complementarity that is required for duplex formation. Stringency can be controlled by temperature, probe concentration, probe length, ionic strength, time, and the like. Preferably, hybridization is conducted under moderate to high stringency conditions by techniques well known in the art, as described, for example in Keller, G. H., M. M. Manak (1987) DNA Probes, Stockton Press, New York, N.Y., pp. 169-170, hereby incorporated by reference. For example, stringent conditions are those that (1) employ low ionic strength and high temperature for washing, for example, 0.015 M NaCl/0.0015 M sodium citrate (SSC); 0.1% sodium lauryl sulfate (SDS) at 50° C., or (2) employ a denaturing agent such as formamide during hybridization, e.g., 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is use of 50% formamide, 5×SSC (0.75 M NaCl, 0.075 M sodium citrate), 50 mM sodium phosphate (pH 6.8), 0.1% sodium pyrophosphate, 5 times Denhardt's solution, sonicated salmon sperm DNA (50 μg/ml), 0.1% sodium dodecylsulfate (SDS), and 10% dextran sulfate at 4° C., with washes at 42° C. in 0.2×SSC and 0.1% SDS.

An example of high stringency conditions is hybridizing at 68° C. in 5×SSC/5×Denhardt's solution/0.1% SDS, followed by washing in 0.2×SSC/0.1% SDS at room temperature. An example of conditions of moderate stringency is hybridizing at 68° C. in 5×SSC/5×Denhardt's solution/0.1% SDS and washing at 42° C. in 3×SSC. The parameters of temperature and salt concentration can be varied to achieve the desired level of complementarity between the capture agent and target. See, e.g., Sambrook et al. (1989) supra or Ausubel et al. (1995) Current Protocols in Molecular Biology, John Wiley & Sons, NY, N.Y., for further guidance on hybridization conditions.

In general, salt and/or temperature can be altered to change stringency. With a capture agent >70 or so bases in length, the following conditions can be used: Low, 1 or 2×SSPE, room temperature; Low, 1 or 2×SSPE, 42° C.; Moderate, 0.2× or 1×SSPE, 65° C.; and High, 0.1×SSPE, 65° C.

“Complex” refers to two or more molecules associated with one another after a successful binding event. As used herein, a complex comprises a capture agent specifically bound or hybridized to a polynucleotide, specifically the target region of the polynucleotide, by Watson-Crick base pairing. To detect a complex, the complex may be labelled with a detectable label, also referred herein as a “label agent”, such as an optically-detectable label. The configuration of capture agent-polynucleotide-label complex is referred to herein as a “labeled complex.”

The term “hybridize” refers to two complementary single-stranded polynucleotide molecules that bind together according to Watson-Crick base-pairing rules to form a double-stranded molecule. For example, the complementary base sequence for 5′-GGAATC-3′ is 3′-CCTTAG-5′. The term “specifically hybridizes” or “specifically binds” refers to a sequence that binds to its complementary sequence with an at least order of magnitude greater frequency compared to a non-specific binding event where the counter sequence is not exactly complementary, or has a binding rate constant at least an order of magnitude better compared to a binding rate constant for any other not exactly complementarity sequence present in the invention.

The term “characterizing” is used broadly herein to refer to analyzing a parameter or variable of one or more distinct polynucleotides, such as identity, quantity, integrity, and/or stability through assessment of one or more signals generated by the complexes. Preferably, the systems and methods provided herein characterize a plurality of parameters, such as identity, quantity, integrity and/or stability of a polynucleotide, such as mRNA. By using any of the methods provided herein as a function of time, stability can be assessed by characterization of the polynucleotide integrity as a function of time and/or storage condition.

As used herein, “sample” refers to a material that may contain a polynucleotide that is to be characterized, such as a material taken from a vaccine development process or an aliquot of a final vaccine, or a material taken from a therapeutic development process or an aliquot of a drug substance or drug product.

“Label” or “label agent” refers to a label that is useful to detect a complex between a target and a nucleic acid capture agent. Any range of label agents may be used, including antibodies such as an antibody against a 5′-cap, and polynucleotides such as nucleic acid oligonucleotides that can hybridize to one or more mRNA targets to form a capture agent-target-label agent complex. Label agents may have a fluorescent, chemiluminescent, or other optical tags to enable downstream detection. Label or label agent may refer to a detection label or a molecule capable of binding to a polynucleotide, including a polynucleotide-capture agent complex, to create a complex that comprises a fluorophore or other optical, electrical, magnetic, electrochemical, or other suitable tag capable of generating a measurable signal that can be detected to aid in characterization. A label may comprise a polynucleotide coupled to a fluorophore or other tag, an antibody coupled to a fluorophore or other tag, and other commonly utilized detection labels known to one skilled in the art.

A “labeled complex” refers to a polynucleotide bound to both a capture agent and bound to a label.

As used herein, “conserved region” refers to a region of a polynucleotide sequence that is identical across multiple polynucleotides, each having overall polynucleotide sequences that are not identical. In other words, at least a portion of a non-conserved region has sequence dissimilarity.

“Integrity” refers to a property of a polynucleotide that is a measure of the intactness, wholeness, or completeness of the target. A polynucleotide that has not degraded and has maintained full sequence has complete integrity. In contrast, a polynucleotide that has degraded, including by loss of at least a portion of the polynucleotide, is characterized as having a loss of integrity. Integrity may be assessed by use of multiple labels to assess whether full-length of the polynucleotide is maintained.

“Stability” refers to a property of a target that is a measure of the intactness, wholeness, or completeness of the target after the target has been exposed to conditions that may affect the integrity, intactness, wholeness, or completeness of the target, including over a period of time. Stability may be assessed by repeating the method of characterizing the polynucleotide over a time course.

“Multiplex characterization” refers to the simultaneous characterization of more than one polynucleotide target on an array.

“Singleplex characterization” refers to the characterization of a single polynucleotide target on an array.

The term “target-specific nucleic acid capture sequence” refers to a polynucleotide capture agent that comprises a sequence that is substantially complementary to a target region sequence of the polynucleotide sequence such that the capture agent binds preferably only to a polynucleotide having a target region sequence with the substantially complementary sequence.

The term “spatial pattern of complexes” refers to more than one complex spatially separated from one another such that the spatial location of the signal produced by each complex enables identification of the target.

The term “threshold value” refers to a selected signal value at which signals exceeding the threshold value are considered a positive and indicates the presence of a labeled complex. Signal values below the threshold value are considered negative, indicating the absence of a target.

The term “positive complex” refers to a complex generating a signal that exceeds a threshold value. The term “negative complex” refers to a complex generating a signal that is below a threshold value.

“mRNA-containing vaccine” or “mRNA-containing therapeutic” refers to a vaccine, drug, biologic, or other therapeutic agent that contains messenger RNA as an active ingredient.

The term “linearizing” refers to any of a variety of chemical or physical methods for denaturing or eliminating or minimizing secondary and tertiary structure of a polynucleotide such as mRNA. Linearizing may promote effective hybridization between two complementary polynucleotides, including by increasing accessibility of the polynucleotide target region to the capture agent.

In one embodiment, the substrate with replicate arrays is manufactured by printing methods conventionally used for manufacturing high- or low-density flat substrate microarrays, for example non-contact microarray printing such as inkjet or piezoelectric printing. Other manufacturing methods are also possible including, but not limited to chemical binding of capture agents to microsphere (“bead”) surfaces in solution or in air, or other array and manufacturing methodologies known in the art.

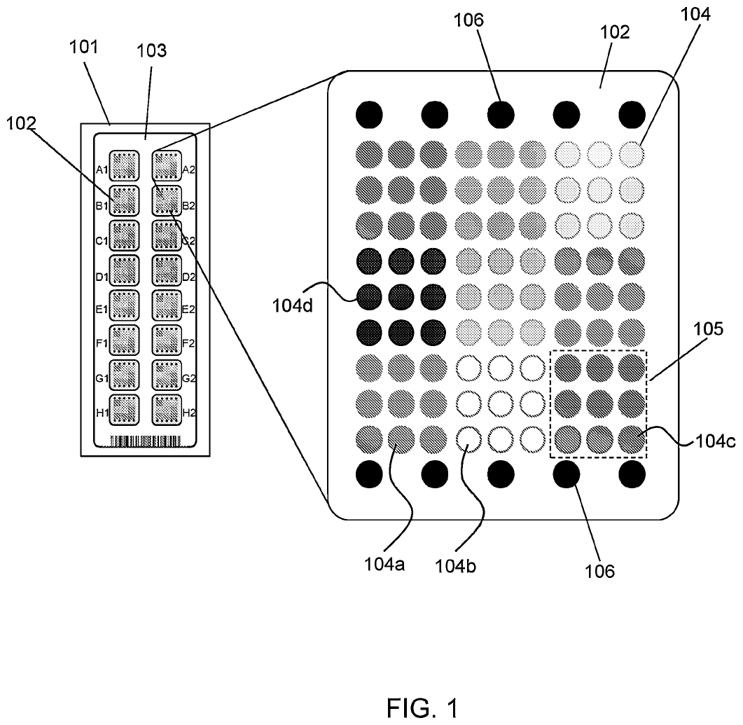

In one embodiment, and as illustrated in FIG. 1 (left panel), multiple replicates of the array 102 may be printed on a substrate 101. The substrate 101 may be approximately 25 mm×75 mm in lateral dimensions and 0.5 mm in thickness. However, in other embodiments substrate 101 may be a different size, for example 25 mm×25 mm in lateral dimensions and 0.5 mm in thickness, or 1 cm by 1 cm in lateral dimensions with thickness 1 mm. In another embodiment, the array 102 may be of a non-rectangular shape and be printed on a non-glass substrate such as a flexible polymer or another flexible substrate that may be produced via roll-to-roll manufacturing. In another embodiment, the array 102 may be printed in the well of a 24-, 48-, 96-, or 384-well plate.

In one embodiment, and as illustrated in FIG. 1, there may be 16 replicate arrays 102 printed on a single substrate 101. However, in other embodiments, there may be alternate numbers and configurations of replicate arrays 102 on the substrate 101.

In one embodiment, the substrate 101 may be additionally outfitted with a gasket 103. In some embodiments, the gasket 103 provides a visual indicator of where the replicate arrays are located on the substrate. In some embodiments, the gasket 103 acts as a reaction vessel that confines a predetermined amount of a vaccine sample or other liquid material to a single array 102.

In one embodiment, and as illustrated in FIG. 1 (right panel), an array 102, has individual spots 104 ranging from 50-400 micrometers in diameter, patterned in a regularly-spaced rectangular array. In another embodiment, some individual areas on the array 102 may be printed with a buffer (“blank” spot) or may be empty and not have a spot printed in the nominal spot location. However, one who is skilled in the art will recognize that many alternative sizes, patterns, shapes and spacings are possible, including with regularly- and irregularly-spaced and rectangular and non-rectangular geometry.

In one embodiment, one or more spots 104 containing multiple copies of a single nucleic acid capture agent 105 may be printed on the array 102, with another spot or other spots containing a different nucleic acid capture agent, as denoted by the different shading for each group of replicate spots shown in FIG. 1. This is further illustrated by spots 104a-104d, with each of a-d (e-i not labelled) intended to reflect the spot comprises different capture agents intended to bind a different target sequence in a polynucleotide. In the illustrated embodiment of FIG. 1, there are nine different capture agents, to detect nine unique polynucleotide sequences. In one embodiment, there may be 9 replicate spots of each capture agent, as illustrated by capture agent 105 in FIG. 1 (each containing replicates as indicated by the nine spots having the same shading). Of course, the methods and arrays are compatible with any number of replicate spots, depending on the application of interest. For example, there may be up to 12 replicate spots of each capture agent (e.g., there would be 12 spots associated with each of the capture agents shaded differently), or as few as 3 replicate spots of each capture agent (e.g., there would be 3 spots associated with each of the capture agents shaded differently). In an embodiment, the group of all replicates of a single capture agent may be referred to as a sub-array (e.g., corresponding to the dashed square outlining the replicates associated with 105). One skilled in the art will recognize that alternative numbers of replicate spots for each capture agent are also possible. The different shading of spots, representing different unique capture agents that each uniquely bind to a different target region of the polynucleotide, provides multiplexing capability. Use of n unique capture agents allows for multiplexing of n unique polynucleotides. In an embodiment, n is between 1 and 12, including between 1 and 9, 1 and 4, and between 2 and 4. As the spatial position of each unique capture agent is known (see, e.g., the different shading of capture agent spots 104), there is no need for unique labels to identify a specific polynucleotide. Instead, the location from which the signal is generated provides the information on the polynucleotide via the capture agent sequence.

In one embodiment, the array 102 may include control spots 106 that may be used as fiducial markers to locate the array and aid in data analysis.

In addition, arrays may be comprised of a plurality of nucleic acid capture agents bound to a plurality of microspheres (a single capture agent per microsphere) that are spatially distinct but free to move relative to one another.

In one embodiment, the capture agent is single stranded oligonucleotide comprised of DNA. In one embodiment, the capture agent is a single-stranded oligonucleotide comprised of DNA bases and one or more non-native bases or modifications. Generally, the capture agent is covalently attached to a substrate surface. Accordingly, in an embodiment the capture agent is modified at one of its termini to allow for covalent attachment of the capture agent to the array surface. The capture agent may be arranged in either a 3′ or 5′ attachment to the substrate. In another embodiment, the capture agent is a single-stranded oligonucleotide comprised of RNA. In another embodiment, the capture agent is a single-stranded oligonucleotide comprised of RNA bases and one or more non-native bases or modifications. In another embodiment, the capture agent is a locked nucleic acid or bridged nucleic acid. In another embodiment, the capture agent is a peptide nucleic acid. In another embodiment, the capture agent is an aptamer. In another embodiment, the capture agent comprises any combination of mixtures of the above exemplified capture agents, depending on the application of interest.

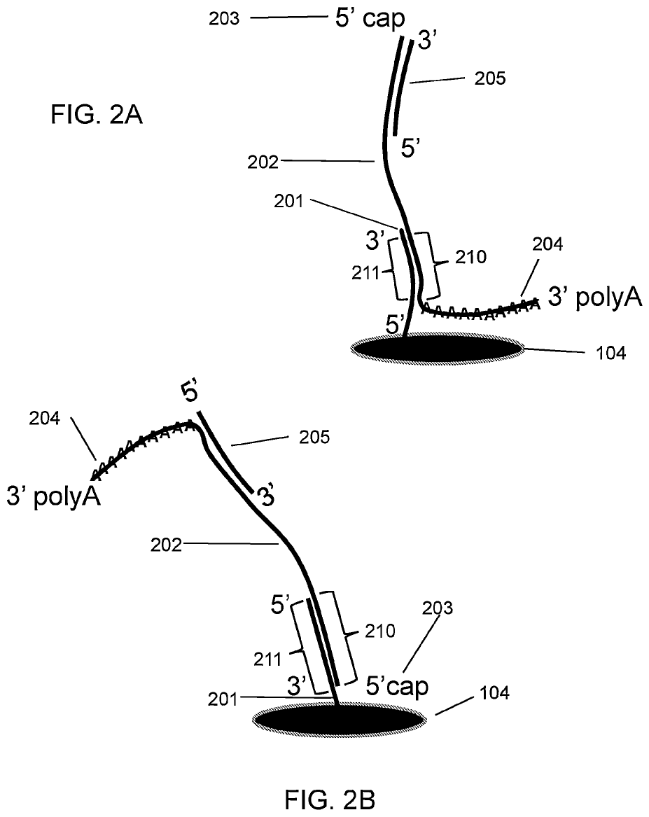

FIG. 2A-2B illustrates the operation of one embodiment of a capture agent 201 in a capture agent spot 104 from the array shown in FIG. 1. In FIG. 2A-2B, single stranded oligonucleotide capture agent 201 is bound to the array surface through a mechanism known to one skilled in the art, such as through a modification to the 5′ end or the 3′ end of the capture agent with an appropriate chemistry to promote covalent attachment to the microarray substrate. A polynucleotide, such as an mRNA construct 202 having a 5′ cap 203, a 3′ poly A tail 204, and a target region 210, may bind to the capture agent 201 through nucleic acid hybridization of complementary sequence portions 210 (polynucleotide target region) and 211 (target-specific nucleic acid capture sequence), with the potential for and orientation (5′ to 3′ or 3′ to 5′) of binding based on the nucleic acid sequence of both the capture agent and the mRNA target. The structure formed by binding of mRNA construct 202 with capture agent 201 is referred herein as a polynucleotide and capture agent complex. The structure formed by the further binding of label 205 is referred herein as a labeled complex. An oligonucleotide label 205 may bind to the polynucleotide 202, including an mRNA construct, through nucleic acid hybridization, with the potential for an orientation of binding based on the nucleic acid sequence of the mRNA target and the label. Alternatively, the label 205 could be an anti-5′ cap antibody capable of binding to the 5′ cap on the mRNA target to provide sequence-independent universal labelling, as well as a characterization directed to the polynucleotide integrity aspect.

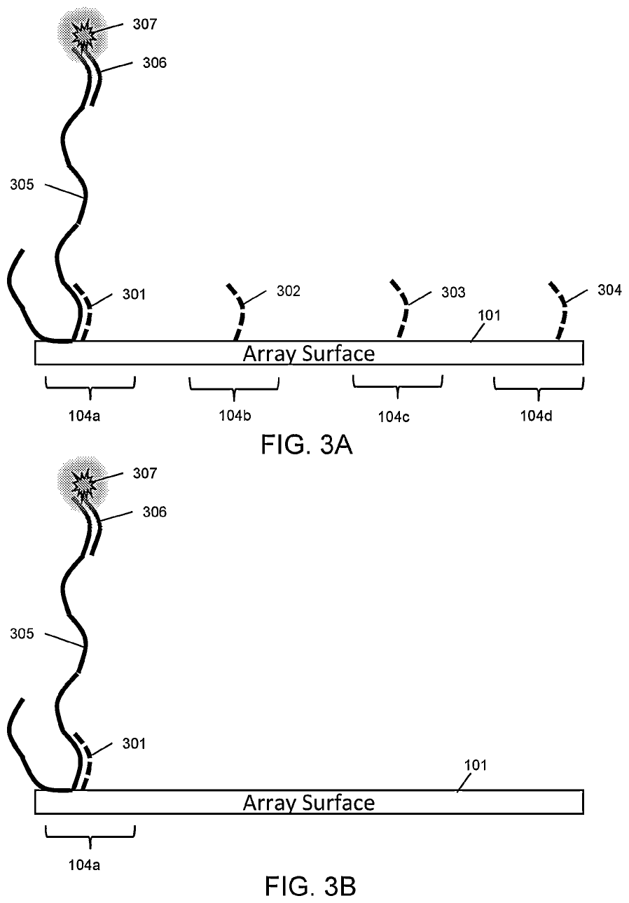

FIG. 3A-3B illustrates the operation of one embodiment of the array shown in FIG. 1 for the singleplex characterization of identity, quantity, or stability of a single mRNA target. Capture agents 301 through 304, corresponding to portions of spots 104a-104d from FIG. 1, are immobilized onto discrete areas or spots of the array surface 101 from FIG. 1. A single mRNA target 305 capable of binding to capture agent 301 based on its complementary sequence is introduced onto the array and hybridizes to capture agent 301 to form a complex. Capture agents 302, 303, and 304 do not bind target mRNA 305 due to the non-complementarity of the capture agent sequences to the target mRNA sequence. The bound target mRNA 305 is then labeled with label 306 that is modified to include a fluorophore 307 capable of generating an optical signal.

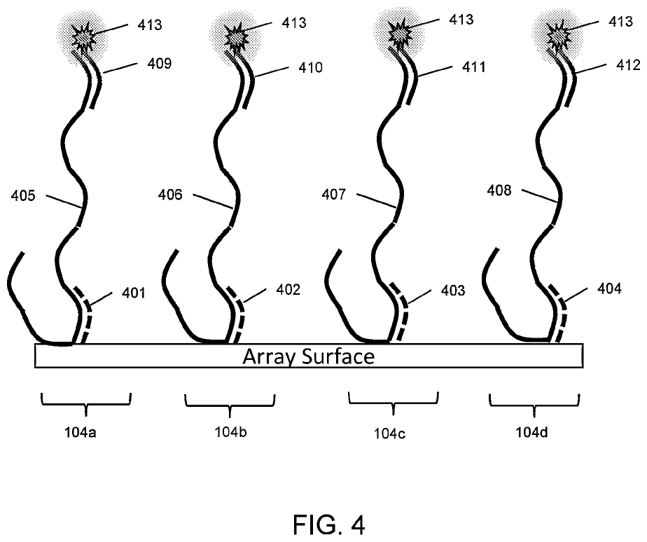

FIG. 4 illustrates the operation of one embodiment of the array shown in FIG. 1 for the multiplex characterization of identity, quantity, or stability of multiple mRNA targets. Capture agents 401 through 404 are immobilized onto discrete areas or spots (labeled as 104a-104d) of the array surface. Multiple unique mRNA targets 405 through 408 are capable of specifically binding to capture agents 401 through 404, respectively, as shown based on sequence complementarity between the capture agent and corresponding target mRNA are introduced onto the array and hybridize to capture agents 401 through 404 as shown. The bound target mRNAs 405 through 408 are then labeled with labels 409 through 412 that are modified to include a fluorophore 413 capable of generating an optical signal. In certain embodiments, labels 409 through 412 are identical in that they are directed to a region of target mRNA that is conserved amongst all target mRNAs present, such as a sequence in the 3′ poly A region or a conserved untranslated region of the target mRNAs. Such labels may be referred herein as a “universal” label in that they will bind more than one unique polynucleotide due to the presence of shared elements or characteristics between target unique mRNAs. In certain embodiments, labels 409 through 412 are all unique in that they are directed to regions of target mRNA that are unique amongst all target mRNAs present, such as a sequence within the coding region. In this context, the label is not universal, but rather is polynucleotide-specific or mRNA target-specific in that the label does not bind to more than one unique polynucleotide.

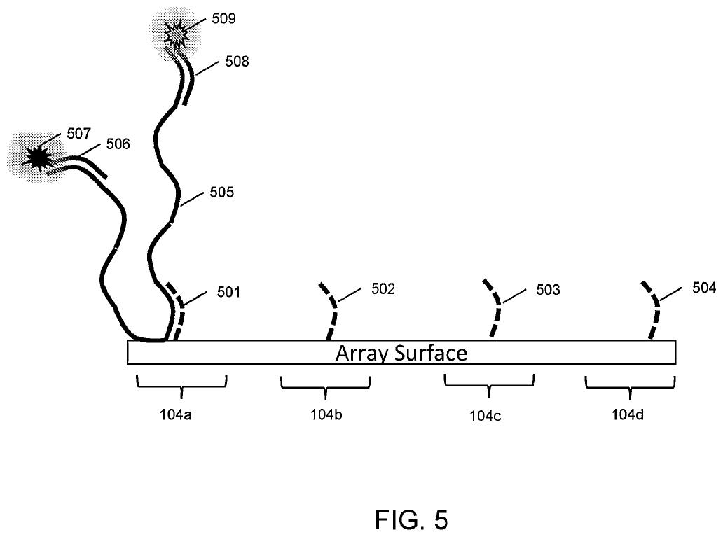

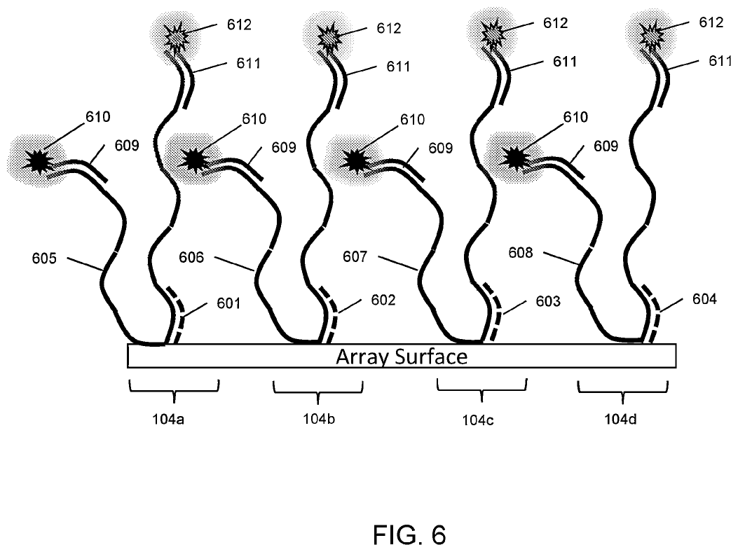

FIG. 5 illustrates the operation of one embodiment of the array shown in FIG. 1 for the singleplex characterization of identity, quantity, or stability of a single mRNA target. Capture agents 501 through 504 are immobilized onto discrete areas or spots of the array surface. A single mRNA target 505 capable of binding to capture agent 501 based on its complementary sequence is introduced onto the array and hybridizes to capture agent 501. Capture agents 502, 503, and 504 do not bind target mRNA 505 due to the non-complementarity of the capture agent sequences to the target mRNA sequence. The bound target mRNA 505 is then labeled with label 506 that is modified to include a fluorophore 507 capable of generating an optical signal over an emission wavelength range to be detected on a first detection channel. Additionally, the bound target mRNA 505 is labeled with label 508 that is modified to include a fluorophore 509 capable of generating an optical signal over a different emission wavelength range to be detected on a second detection channel, therefore creating two unique optical signals from the labeled complex of the single target mRNA 505. Those two unique optical signals provide integrity characterization. If there is degradation at either end of the polynucleotide, FIGS. 5-6 illustrate there will be loss of optical signal associated with labels from lack of binding of labels (FIG. 5: lack of binding of 506 and/or 508; FIG. 6: lack of binding of 609 and/or 611).

FIG. 6 illustrates the operation of one embodiment of the array shown in FIG. 1 for the multiplex characterization of identity, quantity, or stability of multiple mRNA targets. Capture agents 601 through 604 are immobilized onto discrete areas or spots of the microarray substrate. Multiple mRNA targets 605 through 608 capable of binding to capture agents 601 through 604 specifically based on the complementarity of the capture and target sequences are introduced onto the array and hybridize to capture agents 601 through 604. The bound target mRNAs 605 through 608 are then labeled specifically with label 609 that is modified to include a fluorophore 610 capable of generating an optical signal over a first emission wavelength range to be detected on a first detection channel. Additionally, the bound target mRNAs 605 through 608 are also labeled with label 611 that is modified to include a fluorophore 612 capable of generating an optical signal over a second emission wavelength range different from the first to be detected on a second detection channel, therefore creating two unique optical signals from the capture and labeling of each of the single target mRNAs 605 through 608.

In one embodiment, the systems and methods herein may be used to identify the presence of the target, including a plurality of targets, including from a plurality of viral organisms. A fluorescence signal on the array for a specific capture agent or multiple capture agents after a vaccine sample has been applied and label applied to form a labeled complex may be utilized in this embodiment as an identity test for the presence of the desired target in the vaccine sample. Selection of appropriate labels may also characterize the polynucleotide(s) integrity.

In one embodiment, the systems and methods described herein may be used to quantify a target. A calibration curve may be generated by applying dilutions of a standard target to replicate arrays on a substrate. Vaccine samples or other polynucleotide-containing samples with unknown target concentration can also be applied to other replicate arrays on the same substrate or on a different substrate. The method of this embodiment then comprises labeling the bound target on all replicate arrays with an appropriate label agent, determining a relationship between the fluorescence signals generated from the replicate arrays to which dilutions of standard target were applied, and utilizing the fluorescence signals as a calibration curve with which to calculate the target concentrations in the vaccine samples with unknown target concentration. In another embodiment, the analysis of the fluorescence signals from the calibration curve and vaccine samples is automated with a software algorithm.

In one embodiment, the quantification of target can be used to determine concentration of one or more targets during vaccine process development or optimization. This can be achieved by “calibrating” the array with various known concentration levels of polynucleotide(s) and obtaining a calibration curve from a plot of signal intensity as a function of concentration. In another embodiment, the quantification of target can be used to determine in vitro potency or dose of a vaccine, with higher quantities of polynucleotide generally corresponding to higher in vitro potency values or vaccine dose.

In one embodiment, the systems and methods of the current invention may be utilized to simultaneously quantify multiple polynucleotides, also referred herein as multiplex characterization or “multiplex” mode, by use of multiple unique capture agents. Each unique capture agent is configured to uniquely bind to a different polynucleotide target sequence.

In one embodiment, the system and methods described herein may be used to determine the stability of a vaccine sample after a challenge is encountered, such as exposure to heat, pH changes, nucleases, or other degradation protocols known to one skilled in the art. An aliquot of a vaccine sample may be used as a control, and another aliquot of a vaccine sample may be exposed to a degradation protocol such as exposure to 37 C for 7 days. After exposure to the degradation protocol, the control and degraded aliquots of vaccine sample may be analyzed alongside a calibration curve generated by applying dilutions of a standardized target to replicate arrays. The signals resulting from the dilutions of the standardized target may then be used to quantify the target in the control vaccine sample and degraded vaccine sample to measure stability. In some embodiments, the comparative quantification of target in a control and degraded vaccine sample can be used to determine stability of a vaccine. Such information is of importance, particularly for obtaining optimal storage conditions over time.

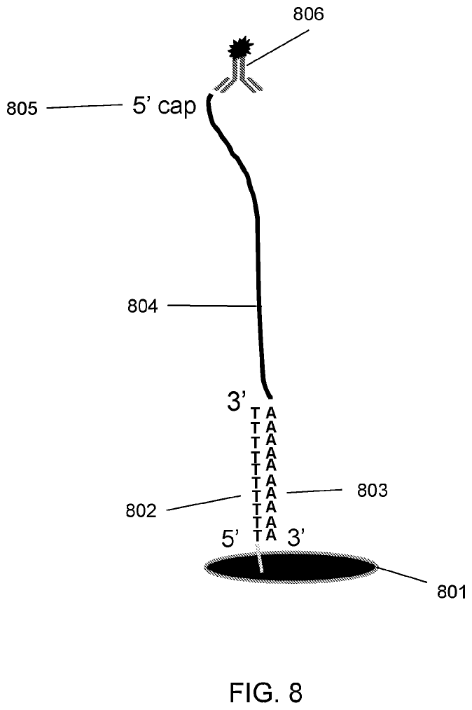

FIG. 8 illustrates one embodiment of the current invention in which a poly T polynucleotide capture agent 802 printed in spot 801 on the array surface is utilized to capture a target mRNA 804 comprising a 5′ cap structure 805, a coding region, and a 3′ poly A tail 803 consistent with features in mRNA vaccine constructs. One of ordinary skill in the art will realize that the target mRNA may include other elements such as 3′ and 5′ untranslated regions. This embodiment enables detection of full-length, intact mRNA by capturing the target mRNA 804 at the 3′ end and using a detection label comprised of an anti-5′ cap antibody 806 to which a fluorophore is conjugated to detect the opposite, 5′ end of the target mRNA.

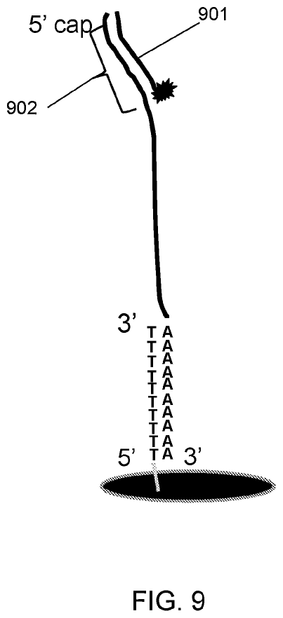

FIG. 9 illustrates another embodiment in which the same poly T capture agent is utilized as shown in FIG. 8, but the detection label 901 is a polynucleotide detection label comprising a sequence that is complementary to the portion of the target mRNA adjacent to the 5′ cap structure 902 to enable label binding at the 5′ end of the target mRNA. This alternative detection labeling methodology also facilitates the measurement of intact, full-length mRNA. The capture and detection strategies in FIGS. 8 and 9, however, do not facilitate separate measurements of intact mRNA for multiple unique target mRNAs, and are therefore useful for singleplex characterization or assessment of total mRNA in a multiplex sample, as the capture agent is universal to all target mRNAs containing a 3′ poly A tail.

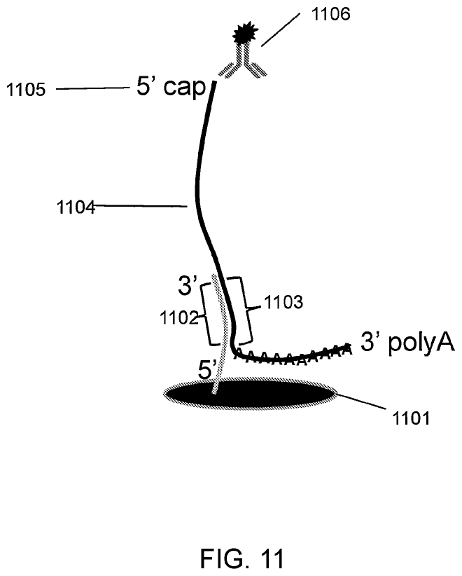

FIG. 11 illustrates another embodiment in which a coding region capture agent 1102 complementary to a portion of the coding region 1103 of a target mRNA 1104 is utilized to capture a target mRNA 1104 comprising a 5′ cap structure 1105, a coding region, and a 3′ poly A tail consistent with features in mRNA vaccine constructs. One of ordinary skill in the art will realize that the target mRNA may include other elements such as 3′ and 5′ untranslated regions. This embodiment facilitates specific detection of a target mRNA provided the capture agent is a target-specific nucleic acid capture sequence designed against a portion of the coding region unique to the mRNA target of interest. A detection label 1106 comprised of an anti-5′ cap antibody to which a fluorophore is conjugated facilitates detection of the 5′ end of the target mRNA.

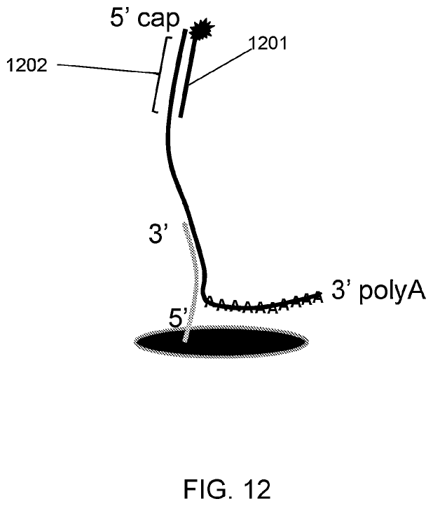

FIG. 12 illustrates another embodiment in which the same coding region capture agent is utilized as shown in FIG. 11, but the detection label 1201 utilized is a polynucleotide detection label comprised of a sequence that is complementary to the portion of the target mRNA adjacent to the 5′ cap structure 1202 to enable label binding at the 5′ end of the target mRNA. This alternative detection labeling technology also enables the specific detection of a target mRNA because a unique portion of the coding region is targeted by the polynucleotide capture agent.

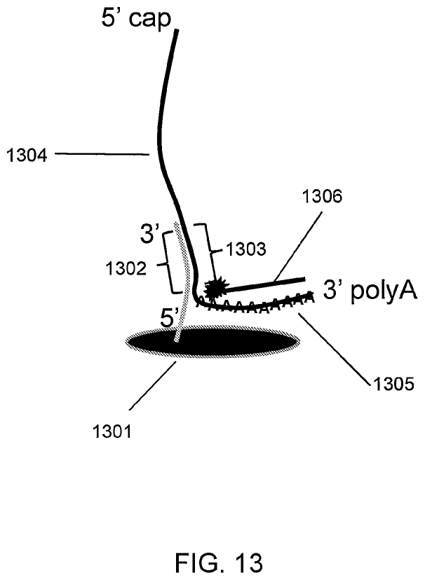

FIG. 13 illustrates another embodiment of the current invention in which the same coding region capture agent 1302 utilized as shown in FIG. 11 is printed in spot 1301 on the array surface, but the detection label 1306 is a poly T polynucleotide detection label complementary to the 3′ poly A tail region 1305 of the mRNA target 1304. This alternative detection labeling technology also enables the specific detection of a target mRNA because a unique portion of the coding region is targeted by the polynucleotide capture agent.

### EXAMPLES

### Example 1

### Array Processing Procedure

In accordance with the methods and systems described herein, a vaccine or therapeutic sample containing one or more polynucleotides of interest that may be comprised of mRNA, RNA, DNA, or other nucleic acid or ribonucleic acids known to one of ordinary skill in the art are mixed with a buffer to promote nucleic acid hybridization based on the nature of the vaccine sample or standard sample to be analyzed.

In one embodiment, the hybridization buffer, alternatively referred to as binding buffer, at 2× concentration contains saline sodium citrate (SSC) at 6× concentration, 4% polyethylene glycol diacrylate (PEGDA) 575, and 4% sodium dodecyl sulfate (SDS).

In another embodiment, the hybridization buffer or binding buffer at 2× concentration contains saline sodium phosphate EDTA (SSPE) at 8× concentration, 0.4% sodium dodecyl sulfate (SDS), and 5× Denhardt's solution.

In another embodiment, the hybridization buffer or binding buffer at 2× concentration contains saline sodium citrate (SSC) at 8× concentration, 0.2% sodium dodecyl sulfate (SDS), and 4× Denhardt's solution.

In another embodiment, the hybridization buffer or binding buffer at 2× concentration contains 8× saline sodium citrate (SSC), 6% BSA, and 0.02% sodium azide,

In another embodiment, the hybridization buffer or binding buffer also includes an additive known to linearize or reduce RNA secondary structure to facilitate microarray hybridization, such as formamide, DMSO, low salt concentration (such as below 0.5M total salt), PEGDA 575, urea, spermidine, detergents, surfactants, or other additives for denaturing or linearizing RNA.

In accordance with the array of the methods and systems described herein, the array is comprised of replicate spots of each of a multiple target-specific nucleic acid capture sequences, with at least one of the target-specific nucleic acid capture sequences complementary to a portion of one of the polynucleotide target sequences, and other polynucleotide capture agents complementary to a portion of another polynucleotide target sequence, so that all relevant polynucleotides in the sample are capable of hybridizing to their respective unique capture agent for independent identification.

The target may be an mRNA construct such as that utilized in an mRNA vaccine. The polynucleotide capture agents may be synthetic DNA oligonucleotides immobilized via a modification to the 5′ end, and the capture agents are immobilized onto epoxide-functionalized glass using a microarray printing method such as contact, non-contact, or piezoelectric methods known to one of ordinary skill in the art. There may be up to 81 replicate spots for each printed capture agents, and more specifically, between 3 and 9 replicate spots for each printed capture agent.

Samples to be analyzed are pre-mixed with buffer as described and are then introduced to the array and incubated for a target-specific time period (e.g., overnight, 2 hours, 1 hour, 30 min, 15 min, 10 min, or 5 min). Samples pre-mixed with hybridization buffer may also be subjected to a short heating step prior to addition to the microarray, such as between 55° C. and 95° C. for 5 minutes, after which it is snap cooled on ice to promote rapid cooling and minimize refolding of RNA in the sample. Sample incubation may occur statically (that is with no mixing), or with mixing on an orbital shaker, rocker, or other appropriate device capable of increasing mass transfer. After incubation of the sample on the array, excess solution is removed from the array(s) by pipette. A solution containing an appropriate label agent for the target being investigated is added to the array(s), and the array(s) are then further incubated for a target-specific time period (e.g. overnight, 2 hours, 1 hour, 30 min, 15 min, 10 min, or 5 min). Similarly, label incubation may occur statically (that is with no mixing), or with mixing on an orbital shaker, rocker, or other appropriate device capable of increasing mass transfer. Excess label agent solution is then removed by pipette, and the substrate washed with an initial wash buffer, with excess wash buffer then removed.

In some embodiments, the substrate may then be washed with a second wash buffer, with excess wash buffer again removed.

One skilled in the art will note that a variety of hybridization and washing procedures may be applied to the current method, including tailored so as to achieved desired stringency conditions to maximize desired signal to noise ratio. Substrates are then dried and imaged using an appropriate imaging system such as a fluorescence microarray imaging system.

### Example 2

### mRNA Identity

The resulting fluorescence image from executing the procedure outlined in Example 1 is obtained and analyzed to characterize polynucleotide(s) identity. Specifically, the resulting signals are extracted from the image and used to detect the presence or absence of each relevant polynucleotide. In a sample containing a single polynucleotide sequence, the signal, signal to background ratio, or other signal metric known to one of ordinary skill in the art is used to compare the signal metric generated by the complex (nucleic acid capture agent known to bind or hybridize to the polynucleotide target sequence) to the signal metric generated on the nucleic acid capture agent(s) known not to bind the target. In this manner, a threshold value is obtained, so that for any given optical image, presence or absence of a polynucleotide is readily determined.

As one example illustrating a singleplex application, FIG. 3B illustrates polynucleotide 305 incubated with an array surface 101 containing a single capture agent 301. In this example, the signal metric generated on Capture Agent 301 after labeling with fluorescent label 306 and 307 will be sufficiently high (such as indicated by a signal to background ratio exceeding a threshold such as 3, 5, or 10) to indicate binding of Target 305, indicating the presence and confirming the identity of Target 305, for example in a sample from a monovalent vaccine bioprocess.

SINGLEPLEX CHARACTERIZATION IN A BIOPROCESS SAMPLE FROM A MULTIVALENT VACCINE: One example illustrating a singleplex application in an eventual multivalent vaccine (such as analysis of a bioprocess sample containing one mRNA target that will become part of a multivalent vaccine), FIG. 3A illustrates target polynucleotide 305 incubated with an array containing four unique polynucleotide capture agents (identified as 301 through 304) printed in spots 104a through 104d on array surface 101, where capture agent 301 is designed to bind target polynucleotide 305 and capture agents 302-304 are designed not to bind polynucleotide target 305, but could bind to other polynucleotide targets (as discussed below for FIG. 4). In this example, the signal metric generated by the positive complex formed on capture agent 301 after labeling with fluorescent label 306 will be sufficiently high (such as indicated by a signal to background ratio exceeding a threshold such as 3, 5, or 10) to indicate binding of Target 305, whereas the signal metrics generated by the negative complexes on capture agents 302 through 304 will be sufficiently low (such as indicated by a signal to background not exceeding, for example, 1.5, 2, or 3) to indicate no binding of target 305. This result would therefore indicate the presence of a positive complex resulting from polynucleotide capture agent 301 and confirm the identity of Target 305, for example, in a monovalent bioprocess sample containing only one target. Alternatively, a similar analysis could be made for a monovalent bioprocess containing target polynucleotides 2, 3, or 4, designed to bind to polynucleotide capture agents 302, 303, and 304, respectively, to confirm identity. This exemplified assay could be of use in a bioprocess where there are four different bioprocesses that make each of target polynucleotides 1-4. One single assay can be used for quality control purposes for each of the four bioprocesses. As discussed below, the same assay can then be used in a multiplex characterization to simultaneously characterize polynucleotides 1-4 in a “complete” product. Of course, for embodiments where only presence of one target polynucleotide is desired, only one polynucleotide capture agent specific to one target region is required. Accordingly, the simplest form of the assay is with one type of polynucleotide capture agent affixed to the substrate surface (FIG. 3B).

MULTIPLEX CHARACTERIZATION IN A BIOPROCESS SAMPLE FROM A MULTIVALENT VACCINE: As illustrated in FIG. 4, the methods and systems are capable of multiplex characterization of a plurality of polynucleotides. Another example consistent with the methods and systems of the current invention, a sample containing four target polynucleotides 1 through 4 (405 through 408), is incubated with an array containing polynucleotide capture agents 1 through 4 (401 through 404), where capture agent 1 401 is designed to bind polynucleotide target 1 405, capture agent 2 402 is designed to bind polynucleotide target 2 406, capture agent 3 403 is designed to bind target polynucleotide 3 407, and capture agent 4 404 is designed to bind target polynucleotide 4 408. Either a universal label that binds all targets present (such as a label that targets a conserved untranslated region, the 5′ cap, or the 3′ poly A tail), or a specific label for each of target polynucleotides 1, 2, 3, and 4 (such as labels intended to bind in the coding region for each target as illustrated by labels 409 through 412 and fluorophore 413 in FIG. 4) are added, generating detectable signals. In this example, the signal metric generated on all four capture agents (401 through 404) indicate the presence and confirm the identity of all 4 target polynucleotides 405 through 408, for example, in a multivalent bulk drug substance or final drug product such as a quadrivalent vaccine.

As another example, multiple capture agents may be designed to bind each target to provide complementary or confirmatory information, thereby further assuring assay reliability and sensitivity.

As another example of a singleplex application, FIG. 5 illustrates a sample containing only target polynucleotide 1 505 is incubated with an array comprised of capture agents 1 through 4 (501 through 504), where capture agent 1 501 is designed to bind a section of the coding region (also referred herein as a “target region” of the polynucleotide—see FIG. 2A-2B element 210) unique to target polynucleotide 1 505, and capture agents 2 through 4 (502 through 504) are designed not to bind target polynucleotide 1 505. A universal label 506 that binds the conserved 3′-poly A tail of target polynucleotide 1 501 is added, with the label containing a fluorophore 507 capable of producing detectable signals on a first fluorescence emission channel. A second universal label 508 that binds either the untranslated region or that binds the 5′ cap, with the label containing a fluorophore 509 capable of producing detectable signals on a second fluorescence emission channel. In this example, the presence and intensity of two different “colors” or channels of fluorescence signal on the locations containing capture agent 1 501 is utilized to confirm the presence and identity of full-length mRNA for target polynucleotide 1 505, for example in a monovalent bulk drug substance or vaccine bioprocess sample. In other words, this example reflects a characterization that is both target polynucleotide identity and integrity. By utilizing a calibration curve, as desired, the characterization can also further comprise target polynucleotide quantity. By characterizing under different conditions and/or over time, an indication of target polynucleotide stability is also obtained.

FIG. 6 is another example of multiplex characterization, from a sample containing target polynucleotides 605 through 608 that is incubated with an array containing capture agents 601 through 604, where capture agent 601 is designed to bind a section of the coding region unique to target polynucleotide 605, capture agent 602 is designed to bind a section of the coding region unique to target polynucleotide 606, capture agent 603 is designed to bind a section of the coding region unique to target polynucleotide 607, and capture agent 604 is designed to bind a section of the coding region unique to target polynucleotide 4 608. A universal label 609 that binds the conserved 3′-poly A tail of all of target polynucleotides 1 through 4 (605 through 608) is added, with the label 609 containing a fluorophore 610 capable of producing detectable signals on a first fluorescence emission channel. A second universal label 611 that binds either the untranslated region that is conserved for all 4 target polynucleotides (605 through 608) or that binds the 5′ cap that is conserved for all targets is added, with the label 611 containing a fluorophore 612 capable of producing detectable signals on a second fluorescence emission channel. In this example, the presence and intensity of two different “colors” or channels of fluorescence signal on each capture agent are utilized to confirm the presence and identity of full-length polynucleotide for each target, for example in a multivalent bulk drug substance or final drug product such as a quadrivalent mRNA vaccine.

### Example 3

### Quantification of mRNA

The methods and systems described herein can quantify of one or more target polynucleotides in a vaccine or therapeutic sample by using one or more calibration curves from one or more arrays and one or more standardized polynucleotide dilutions at known concentrations. For example, by running the procedure outlined in Example 1 for various known standardized polynucleotide(s) concentrations. Signal metrics from the resulting fluorescence images from the arrays contacted with standardized polynucleotide dilutions at known concentration are then extracted for each of the polynucleotide capture agents on the array and plotted as a function of the known concentration of the standardized polynucleotide(s) in each dilution to create a calibration curve for each standardized polynucleotide.

One or more arrays are then contacted with a sample containing one or more unique target polynucleotides and processed according to the procedure in Example 1. Signal metrics from the resulting fluorescence images for each polynucleotide capture agent are then compared to the calibration curve for each standardized polynucleotide to back-calculate unknown concentration of the one or more unique target polynucleotides in the sample.

The calibration curve can be best-fit to a linear function or other appropriate functions known to one of ordinary skill in the art such as a 4-parameter logistic function, a 5-parameter logistic function, quadratic function, and other functions utilized for the analysis of binding data. With such a calibration curve, concentration is determined based on the measured signal from a sample having to-be-characterized target polynucleotide(s).

The sample may contain a single target polynucleotide such as that encountered in bioprocess applications or monovalent bulk drug substance, or the same may contain multiple polynucleotide targets such as those encountered in a multivalent bulk drug substance or final drug product such as a multivalent mRNA vaccine.

SINGLEPLEX quantification: As an example of singleplex quantification using a method and/or system described herein, a sample containing target polynucleotide 1 305 is incubated with an array containing capture agents 1 through 4 (301 through 304), where capture agent 1 301 is designed to bind target polynucleotide 1 305 and capture agents 2-4 (302 through 304) are designed not to bind target polynucleotide 1 305. In addition, multiple serial dilutions of a standardized polynucleotide with known quantity of intact polynucleotide 1 305 are added to replicate arrays to generate a calibration curve. A label 306 and 307 designed to bind target polynucleotide 1 305 is added to all arrays for both the standards and sample and used to generate a calibration curve. The calibration curve correlating known concentration of target polynucleotide 1 305 in the standard to signal generated by the label 306 and 307 is then utilized to determine the concentration of target polynucleotide 1 305 in the sample. This result would therefore both confirm the identity of target polynucleotide 1 305, and additionally quantify the amount of target polynucleotide 1 305 present, for example in a monovalent bioprocess sample containing only one target. Alternatively, a similar analysis could be made for a monovalent bioprocess containing targets designed to bind capture agents 2 through 4 (302 through 304) to confirm identity and measure unknown target concentration. Accordingly, any of the methods described herein relates to a single array that can be used for quality control in a bioprocess that involves a plurality of distinct bioprocesses that each related to making a distinct target polynucleotide that is then combined to form a product comprising a plurality of target polynucleotides. This provides the functional benefit of ease of handling/tracking and can provide substantial cost and time efficiencies.

MULTIPLEX quantification: As another example of multiple quantification using a method and/or system provided herein, a sample containing four targets, polynucleotides 1 through 4 (405 through 408), is incubated with an array containing capture agents 1 through 4 (401 through 404), where capture agent 1 401 is designed to bind target polynucleotide 1 405, capture agent 2 402 is designed to bind target polynucleotide 2 406, capture agent 3 403 is designed to bind target polynucleotide 3 407, and capture agent 4 404 is designed to bind target polynucleotide 4 408. In addition, multiple serial dilutions of a quadrivalent standard with known quantity of intact target polynucleotide 1 405, target polynucleotide 2 406, target polynucleotide 3 407, and target polynucleotide 4 408 are added to replicate arrays to generate a calibration curve for all four target polynucleotides. Either a universal label that all target polynucleotides present, such as a label that targets a conserved untranslated region, the 5′ cap, or the 3′ poly A tail (where 409 through 412 would have identical sequences and the same fluorophore 413), or specific labels 409 through 412 (where each has a unique sequence but the same fluorophore 413) for each of target polynucleotides 1, 2, 3, and 4 405 through 408 (such as labels intended to bind in the coding region for each target) are added, generating detectable signals. The four calibration curves correlating known concentration of each standardized polynucleotide to signal generated by the corresponding label are then utilized to determine the concentration of all four target polynucleotides 405 through 408 in the sample. This result would therefore both confirm the identity of all four target polynucleotides 405 through 408, but additionally quantify the amount of each of the four target polynucleotides 405 through 408 present in the sample, for example in a multivalent bulk drug substance or final drug product such as a quadrivalent mRNA vaccine.

Multiplexed quantification using 2 fluorophores: As another example, a sample containing target polynucleotides 1 through 4 (605 through 608) is incubated with an array containing capture agents 1 through 4 (601 through 604), where capture agent 1 601 is designed to bind a section of the coding region unique to target polynucleotide 1 605, capture agent 2 602 is designed to bind a section of the coding region unique to target polynucleotide 2 606, capture agent 3 603 is designed to bind a section of the coding region unique to target polynucleotide 3 607, and capture agent 4 604 is designed to bind a section of the coding region unique to target polynucleotide 4 608. In addition, dilutions of a quadrivalent standard with known concentrations of polynucleotides 1, 2, 3, and 4 (605 through 608) known to be full length is added to replicate arrays. A universal label 609 that binds the conserved 3′-poly A tail of all of target polynucleotides 1 through 4 (605 through 608) is added to the arrays containing the standard and the sample, with the label containing a fluorophore 610 capable of producing detectable signals on a first fluorescence emission channel. A second universal label 611 that binds either the untranslated region that is conserved for all 4 target polynucleotides (605 through 608) or that binds the 5′ cap that is conserved for all Polynucleotide 605 through 608 is added simultaneously, with the second universal label 611 containing a fluorophore 612 capable of producing detectable signals on a second fluorescence emission channel. The individual fluorescence intensities of the standards are used to create calibration curves for each target in the standard and can then be used to quantify the unknown concentration of target polynucleotides 1-4 (605 through 608) in the sample. In addition, the ratio of fluorescence signals on the two channels for the standard can be compared to the ratio of fluorescence signals generated on each channel for the sample to determine the relative amount of full-length mRNA for each of target polynucleotides 1 through 4 (605 through 608) in the sample, for example in a multivalent bulk drug substance or final drug product such as a quadrivalent mRNA vaccine.

### Example 4

### Identity of mRNA in Influenza Vaccine Samples

The methods and systems are useful for vaccines against any number of infectious pathogens, and are particularly useful for characterizing and assessing mRNA vaccines. Referring to FIG. 3A-3B, a sample such as a vaccine bioprocess sample or monovalent bulk drug substance may contain a single influenza mRNA construct (polynucleotide) 305 which contains the coding region for the hemagglutinin (HA) protein for an influenza A/H1 strain present in a vaccine sample. The A/H1 monovalent vaccine sample is contacted with an array containing four capture agents 301 through 304, where each of the four capture agents 301 through 304 is designed to bind a portion of the mRNA coding region for each of the four mRNA polynucleotides that may be a component of a seasonal influenza vaccine, namely A/H1 301, A/H3 302, B/Yamagata 303, and B/Victoria 304. Label 306 could be either a universal label that binds to all four influenza mRNA polynucleotides (such as a label that targets a conserved portion of the untranslated region, the 5′ cap, or the 3′ poly A tail) or a specific label intended to bind a separate specific portion of the coding region of the target (target portion of the polynucleotide). In this example, detectable signals (such as those with a signal to background ratio exceeding 3, or 5, or 10) are generated on only the capture agent specific for influenza A/H1 301. The signal metrics generated on the capture agents specific for influenza A/H3 302, influenza B/Victoria 303, and influenza B/Yamagata 304 produce detectable signals that are below a threshold cutoff, such as a signal to background ratio less than 3, 2.5, 2.0, or 1.5. The presence of signal above a threshold on the A/H1 capture sequence 301 and absence of detectable signals above a threshold for the other capture agents 302 through 304 indicate the presence and identity of the influenza A/H1 mRNA construct 305.

In another example, the influenza vaccine sample may be a monovalent vaccine sample containing an mRNA polynucleotide including the coding region HA from influenza A/H1, influenza A/H3, influenza B/Yamagata, or influenza B/Victoria.

In another example, the influenza vaccine sample may additionally contain an mRNA polynucleotide that includes the coding region for the influenza neuraminidase (NA) protein for the strain of interest to enable identity confirmation of both the HA and NA mRNA polynucleotides.

As another example, the methods and systems provided herein may be used in a sample that contains many influenza polynucleotides, including up to 4 influenza polynucleotides (405 through 408) that are mRNA polynucleotides which contains the coding region for the hemagglutinin (HA) proteins for influenza A/H1, influenza A/H3, influenza B/Yamagata, and influenza B/Victoria strains that are included in a seasonal influenza vaccine sample. The vaccine sample is contacted with an array containing four capture agents (401 through 404), where capture agent 1 401 is designed to bind a portion of the mRNA coding region for an influenza A/H1 mRNA polynucleotide 405, capture agent 2 402 is designed to bind a portion of the mRNA coding region for an influenza A/H3 mRNA polynucleotide 406, capture agent 3 403 is designed to bind a portion of the mRNA coding region for an influenza B/Yamagata mRNA polynucleotide 407, and capture agent 4 404 is designed to bind a portion of the mRNA coding region for an influenza B/Victoria mRNA polynucleotide 408. A label that is either universal for all four influenza mRNA constructs (such as a label that targets a conserved portion of the untranslated region, the 5′ cap, or the 3′ poly A tail, where 409 through 412 would have identical sequences and the same fluorophore 413, or is specific and intended to bind a separate specific portion of the coding region for each target (in which 409 through 412 would have unique sequences and the same fluorophore 413) are added, generating detectable signals. The signal metric generated on all four capture agents 401 through 404 indicate the presence and confirm the identity of mRNAs for influenza A/H1N1, influenza A/H3N2, influenza B/Victoria, and influenza B/Yamagata in a multivalent bulk drug substance or final drug product such as a quadrivalent seasonal influenza vaccine.

In another example, the influenza vaccine sample may also contain four additional mRNA polynucleotides containing the coding regions for the influenza neuraminidase (NA) protein for one of influenza A/H1N1, influenza A/H3N2, influenza B/Yamagata, and influenza B/Victoria to enable identity confirmation of both the HA and NA mRNA polynucleotides in a quadrivalent influenza vaccine sample. Because in a vaccine the mRNA polynucleotide sequence will be known, suitable capture agents are provided to facilitate multiplex characterization of any number of mRNA polynucleotides in the sample.

In another example the influenza vaccine sample may be a trivalent influenza vaccine sample that only contains a single influenza B strain, and the array may only contain HA and/or NA capture agents specific for a relevant influenza A/H1N1 strain, influenza A/H3N2 strain, and a single influenza B strain that depends on vaccine composition.

In this manner, the methods and systems provided herein are compatible with any number of multivalent vaccines, including vaccines against seasonally-varying viruses, and different distinct pathogens, such as influenza and/or coronavirus and/or any other type of virus amendable to vaccination via an mRNA vaccine. Capture agents are provided against each of the unique mRNA polynucleotides desirably in the multivalent vaccine. The platform is similarly generally applicable to any number of mRNA therapeutics, wherein on or more mRNA polynucleotides are to-be-provided to a patient in need of the mRNA therapy.

### Example 5

### Quantification in an Influenza Vaccine Sample; Including with the Use of Capture Sequences Based on Conserved HA/NA Sequences

The methods and systems provided herein are compatible with an influenza vaccine sample containing a single mRNA polynucleotide 305 that contains the coding region for the HA protein of a relevant influenza A/H1 strain. The sample is incubated with an array containing capture agents 1 through 4 301 through 304, where capture agent 1 301 is designed to bind a portion of the coding region for the HA protein of a relevant influenza A/H1 strain, capture agent 2 302 is designed to bind a portion of the coding region for the HA protein of a relevant influenza A/H3 strain, capture agent 3 303 is designed to bind a portion of the coding region for the HA protein of a relevant influenza B/Yamagata strain, and capture agent 4 304 is designed to bind a portion of the coding region for the HA protein of a relevant influenza B/Victoria strain. In addition, multiple serial dilutions of a standard with known quantity of intact influenza A/H1 mRNA polynucleotide are added to replicate arrays to generate a calibration curve. A label 306 designed to bind the influenza A/H1 mRNA construct 305 with attached fluorophore 307 is added to all arrays for both the standards and sample and used to generate a calibration curve. The calibration curve correlating known concentration of influenza A/H1 mRNA polynucleotide 305 in the standard to signal generated by the label is then utilized to determine the concentration of influenza A/H1 mRNA polynucleotide 305 in the sample. This result would therefore both confirm the identity of the influenza A/H1 mRNA polynucleotide 305, but additionally quantify the amount of influenza A/H1 mRNA construct 305 present, for example in a monovalent bioprocess sample containing only one polynucleotide. Alternatively, a similar analysis could be made for a monovalent bioprocess containing an influenza A/H3 mRNA polynucleotide, an influenza B/Yamagata mRNA polynucleotide, or an influenza B/Victoria mRNA polynucleotide to confirm identity and measure concentrations in other monovalent influenza vaccine samples with unknown concentration.

The methods and systems are compatible with a quadrivalent influenza mRNA vaccine sample containing four mRNA polynucleotides for relevant strains of influenza A/H1 405, influenza A/H3 406, influenza B/Yamagata 407, and influenza B/Victoria 408. The sample with the four polynucleotides is incubated with an array containing capture agents 1 through 4 (401 through 404), where capture agent 1 401 is designed to bind the coding region of the influenza A/H1 mRNA polynucleotide 405, capture agent 2 402 is designed to bind the coding region of the influenza A/H3 mRNA polynucleotide 406, capture agent 3 403 is designed to bind the coding region of the influenza B/Yamagata mRNA polynucleotide 407, and capture agent 4 404 is designed to bind the coding region of the influenza B/Victoria mRNA polynucleotide 408. In addition, multiple serial dilutions of a quadrivalent influenza mRNA vaccine standard with known quantity of each of the 4 mRNA polynucleotides are added to replicate arrays to generate a calibration curve for all four mRNA polynucleotides. A label that universally binds all target polynucleotides present (such as a label that targets a conserved untranslated region, the 5′ cap, or the 3′ poly A tail, in which labels 409 through 412 are the same and have identical sequences), or a specific label for each mRNA polynucleotide (such as labels intended to bind in the coding region for each target, in which labels 409 through 412 would be unique to each target polynucleotide and therefore have unique sequences) are added, generating detectable signals from the associated single fluorophore 413. The four calibration curves correlating known concentration of each mRNA polynucleotide in the standard to signal generated by the label for each mRNA polynucleotide are then utilized to determine the concentration of each of the 4 mRNA polynucleotides in the influenza vaccine sample. This result would therefore both confirm the identity of all four mRNA polynucleotides (405 through 408), but additionally quantify the amount of each of the four mRNA polynucleotides 405 through 408 present in the sample, for example in a multivalent bulk drug substance or final drug product such as a quadrivalent influenza vaccine.

As another example, a quadrivalent influenza mRNA vaccine sample containing four mRNA polynucleotides for relevant strains of influenza A/H1 605, influenza A/H3 606, influenza B/Yamagata 607, and influenza B/Victoria 608, is incubated with an array containing capture agents 1 through 4 (601 through 604), where capture agent 1 601 is designed to bind the coding region of the influenza A/H1 mRNA polynucleotide 605, capture agent 2 602 is designed to bind the coding region of the influenza A/H3 mRNA polynucleotide 606, capture agent 3 603 is designed to bind the coding region of the influenza B/Yamagata mRNA polynucleotide 607, and capture agent 4 604 is designed to bind the coding region of the influenza B/Victoria mRNA polynucleotide 608. In addition, multiple serial dilutions of a quadrivalent influenza mRNA vaccine standard with known quantity of each of the 4 mRNA polynucleotides (601 through 604) are added to replicate arrays to generate a calibration curve for all four target mRNA polynucleotides. A universal label 609 that binds the conserved 3′-poly A tail of all four mRNA polynucleotides is added to the arrays containing the standards and the sample, with the label containing a fluorophore 610 capable of producing detectable signals on a first fluorescence emission channel. A second universal label 611 that binds either the untranslated region that is conserved for all 4 mRNA polynucleotides or that binds the 5′ cap that is conserved for all four mRNA polynucleotides is added simultaneously, with the second universal label 611 containing a fluorophore 612 capable of producing detectable signals on a second fluorescence emission channel. The individual fluorescence intensities of the standards are used to create calibration curves for each polynucleotide in the standard and can then be used to quantify the unknown concentration of all 4 mRNA polynucleotides 605 through 608 in the sample. In addition, the ratio of fluorescence signals on the two channels for the standard can be compared to the ratio of fluorescence signals generated on each channel for the sample to determine the relative amount of full-length mRNA for each of polynucleotides 1 through 4 in the sample 605 through 608, for example in a multivalent bulk drug substance or final drug product such as a quadrivalent vaccine.

The methods and systems may be directed to conserved sequences, including conserved regions for influenza HA and NA sequences. Each of the influenza-directed capture agents on the microarray are designed specifically to bind a region of the mRNA polynucleotide that codes for a region of the influenza hemagglutinin (HA) protein that is conserved at the nucleic acid level across a wide variety of influenza strains for each of the influenza A/H1, influenza A/H3, influenza B/Yamagata, and influenza B/Victoria components in an influenza seasonal vaccine sample. A quadrivalent influenza mRNA vaccine sample is added to the array, and the mRNA polynucleotides (more specifically, the target regions of the polynucleotides) bind to the capture agents on the array and are subsequently labeled to enable identity testing and quantification of the mRNA polynucleotides in the vaccine sample. Because the capture agents on the microarray are DNA sequences that are conserved amongst a wide variety of strains within each of the four subtypes or lineages included in a quadrivalent vaccine sample, the microarray would enable identity and or quantitation of all 4 component polynucleotides as a function of time without the need to update the capture agents seasonally as the recommended vaccine sequences were updated. In addition, this design enables the microarray to be utilized regardless of manufacturer, as while the exact strains and mRNA construct sequences may not be conserved amongst all influenza mRNA vaccine manufacturers, the conserved regions targeted by the capture agents should be conserved amongst all strains that may be included. This avoids the need of having to yearly update the assay, with attendant increase in efficiency and decrease in costs.

In another example, the array of the current systems and methods may additionally contain capture agents designed specifically to bind a region of an mRNA construct that code for a region of the influenza neuraminidase (NA) protein that is conserved at the nucleic acid level across a wide variety of influenza strains for each of the influenza A/N1, influenza A/N2, influenza B/Yamagata, and influenza B/Victoria components in an influenza seasonal vaccine sample.

In another example, the influenza vaccine sample may also contain four additional mRNA constructs containing the coding regions for the influenza neuraminidase (NA) protein for one of influenza A/H1N1, influenza A/H3N2, influenza B/Yamagata, and influenza B/Victoria to enable identity confirmation of both the HA and NA mRNA constructs in a quadrivalent influenza vaccine sample.

In another example the influenza vaccine sample may be a trivalent influenza vaccine sample that only contains a single influenza B strain, and the array may only contain HA and/or NA capture agents specific for a relevant influenza A/H1 strain, influenza A/H3 strain, and a single influenza B strain that depends on vaccine composition.

### Example 6

### Combination Vaccine Against Different Unique Viruses

In another example, the method may comprise a microarray containing a collection of capture agents capable of specifically detecting more than one target polynucleotide, with each capture agent designed against a different respiratory pathogen or vaccine component, and the vaccine sample applied to the array may be a combination vaccine containing target mRNA polynucleotides for more than one pathogen, such as a combination vaccine for more than one respiratory virus such as influenza and coronavirus, or influenza and RSV, or other combinations of respiratory viruses suitable for combining into a single vaccine. Labels specific to each target facilitates simultaneous characterization, including identity and quantification, of more than one pathogen mRNA in a combination vaccine.

### Example 7

### Quality-Control Applications

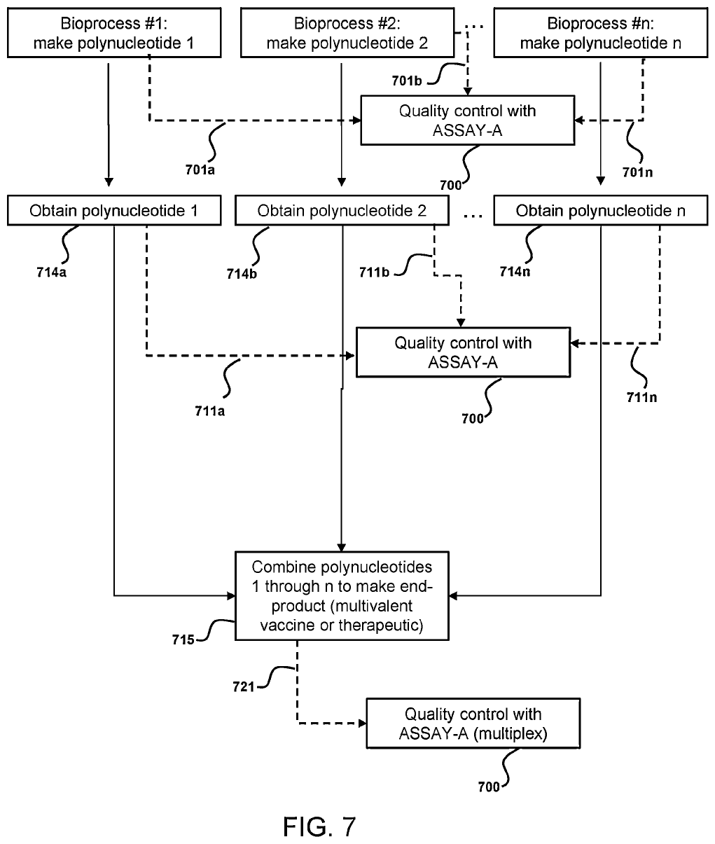

FIG. 7 is a flow-chart summary of one bioprocess used to make a multivalent vaccine to illustrate the advantages of the instant methods and assay systems 700 with respect to quality-control applications (and, similarly, for bioprocess optimization). For a multivalent vaccine containing a number “n” unique mRNA polynucleotides, each of the n polynucleotides must be made or obtained from another manufacturer. The methods and assays provided herein may be used, as reflected by steps 701a, 701b through 701n, to ensure the bioprocess #1 through bioprocess #n are providing the desired polynucleotides. As soon as a characterization by the instant methods and/or assays, indicated by 700, indicates a poor-quality state, such as low number, absence, or degradation, that particular bioprocess can be halted, analyzed and corrected. In a similar manner, the methods and assays provided herein can be readily used to inform a bioprocess optimization, to maximize production and purity of the mRNA polynucleotides. Steps 711a, 711b, 711n indicate that the methods and assays provided herein may be used at any time before combination of the n mRNA polynucleotides that are combined to make the desired end-product 715 by combining the unique polynucleotides 714a, 714b . . . 714n. This could be applicable for situations where one or more of the polynucleotides have been stored awaiting for any one or more other components of the product to be obtained. Steps 701 and 711 reflect the situation that the method and assay may have a singleplex aspect.

An advantage of the instant methods and assay systems is the rapid time to result, such as 2 hours or less as described previously, that enable characterization of the bioprocess closer to the process itself so as to enable elimination of the need to send samples to a centralized laboratory to be run in a queue with a slow turnaround time on the order of one to two weeks.

Finally, step 721 reflects the assays and methods may also be used to characterize the end-product, in a multiplex fashion for simultaneously assessing a plurality of n polynucleotides in the multivalent vaccine or therapeutic. Generally, n is less than or equal to 12, less than or equal to 9, less than or equal to 4, or less than or equal to 3, including between 3 and 9. One particular benefit of the methods and assays provide herein, is that one type of assay 700 (identified as “ASSAY-A”) can be used for all the different quality control assays, including singleplex for different unique polynucleotides (step 701a-701n) and for stability characterization (steps 711a-711n) and multiplex (step 721), as reflected by the same element number 700 at the different locations in the flow-chart. Of course, as desired, different assays, such as a plurality of different singleplex assays, each having only one unique capture agent, may be used. Then, a multiplex assay having a plurality of n-different capture agent types may be used. The advantage of a single assay is for ease of tracking, handling and analyzing, with attendant cost savings.

### Example 8

### Detection of Intact, Full-Length mRNA Construct (FIGS. 8-10)

FIG. 8 is a schematic of an embodiment that facilitates the capture of a full-length, intact mRNA construct from the 3′-polyA tail to the 5′ cap. A polynucleotide capture agent is printed in a microarray spot or location 801 comprising a synthetic DNA nucleotide with a polyT sequence 802 immobilized via its 5′ end that is complementary to the 3′ polyA tail 803 of an mRNA construct 804. The mRNA construct 804 has a 5′ cap structure 805 that can be labeled using a label agent comprised of a fluorescently-conjugated anti-5′ cap antibody 806. This detection scheme ensures detection of the entire intact mRNA construct from end to end, enabling concentration determination of intact mRNA in a monovalent sample (containing only a single mRNA construct), and also facilitating total intact mRNA concentration in a multivalent mixture (containing multiple unique mRNA constructs). This detection scheme does not facilitate identity or concentration determination of individual mRNA constructs in a multivalent mixture, however, as all mRNA constructs would likely have identical 3′ polyA tail and 5′ cap structures and would therefore not be able to be discriminated from one another.

A number of anti-5′ cap antibodies are available including those that may be specifically produced or are already available, such as those available from MBL International Company (RN016M and RN017M) or Sigma (MABE419). One skilled in the art will realize that while an anti-5′ cap antibody labeling detection scheme may be utilized, this same effect may also be achieved utilizing the detection scheme shown in FIG. 9. FIG. 9 shows the same schematic described in FIG. 8, except that instead of an anti-5′ cap antibody label 806, the detection label 901 is a fluorescently-conjugated polynucleotide sequence designed to be complementary to the sequence region immediately adjacent to the 5′ cap 902 on the mRNA construct.

One skilled in the art will also realize the entire schematics in FIGS. 8 and 9 could be inverted such that the label agent (that is, either anti-5′ cap antibody 806 or fluorescently-conjugated polynucleotide sequence 901) could be alternatively immobilized onto the microarray surface without the fluorescent molecule and that the capture agent (that is, the polyT polynucleotide sequence 802) could instead be utilized as a label agent in solution by conjugating it to a fluorescent molecule of interest.

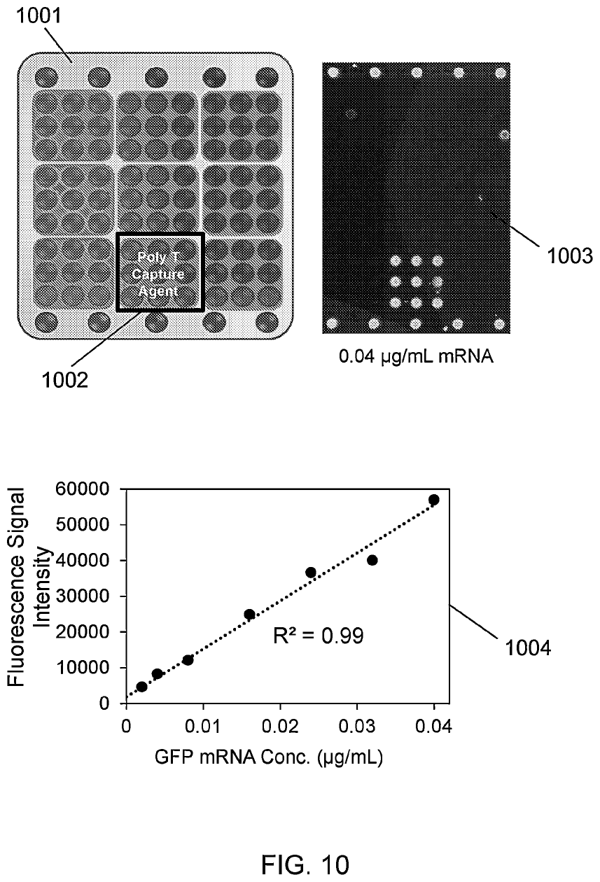

In FIG. 10, the microarray 1001 is comprised of 9 polynucleotide capture agents, each printed in 9 replicate spots, comprising a polyT capture agent 1002 of 30 nt in length with a 5′-amino-C6 modification to enable efficient coupling to the epoxide-functionalized glass microarray surface. The microarray 1001 is printed in 16 replicates onto a single epoxide-functionalized glass slide, and a 16-well silicone gasket is then adhered to create an individual ˜50 μL reaction volume for each microarray. Alternatively, the polyT polynucleotide capture agent could be shorter in length, such as 25 nt or 20 nt, or other lengths so long as the result is efficient room temperature hybridization.

An mRNA construct including the open reading frame (ORF) or coding sequence for green fluorescent protein (GFP) was utilized. This mRNA construct does not include an untranslated region (UTR) but does include a ˜300 nt polyA tail similar to a typical mRNA construct. An initial 1:10 dilution of the stock mRNA was prepared in nuclease-free water, and then further diluted using a 2× binding buffer consisting of 8× saline sodium citrate (SSC), 4× Denhardt's reagent, and 0.2% sodium dodecyl sulfate (SDS), with subsequent serial dilutions made in 2× binding buffer (all samples at final 1× binding buffer concentration). Each dilution was applied to a separate replicate microarray 301 (FIG. 3A-3B) for 60 minutes on a small rotation diameter orbital shaker to facilitate hybridization between the mRNA target and the capture agents on the microarray. The mRNA target-containing solutions were then removed, and the slides were washed with a wash solution containing 2× SSC and 0.1% SDS for 1 minute on the same orbital shaker. A fluorescently conjugated anti-5′ cap detection antibody at 10 ug/mL in final buffer concentration of 1× mRNA binding buffer was then added to all microarrays under analysis and allowed to incubate on the orbital shaker for 30 minutes. After detection label incubation, the slides were washed first with a solution of 2× SSC and 0.1% SDS, followed by two serial washes with 0.2× SSC. The slides were then imaged with the VaxArray® Imaging System (InDevR, Boulder, CO), and the fluorescence intensities on the polyT capture agent for the serial dilution subsequently analyzed. Representative fluorescence image 1003 shows an example image of a solution containing 0.04 μg/mL of mRNA hybridized to the polyT capture agent 1002 and subsequently labeled with an anti-5′ cap antibody.

Plot 1004 shows the linear fluorescence response obtained on the microarray for a serial dilution of the GFP mRNA construct, with a linear regression through the 7 dilutions producing a correlation coefficient (R²) of 0.99. Importantly, these data indicate successful capture of full-length, intact mRNA because the capture event occurs at the 3′ end of the mRNA construct via the 3′ polyA tail sequence, and the detection labeling event occurs at the opposite 5′ end of the mRNA construct via the 5′-cap structure. In addition, the linearity of response indicates that the amount of full-length, intact mRNA can not only be detected, but can be quantified. Quantification of a sample containing an unknown amount of full-length, intact mRNA would be quantified against an appropriate full-length, intact standard.

Based on these data, the lower limit of quantification based on an estimation of ˜3× background signal is likely in the low ng/mL range. This detectability is certainly relevant for mRNA vaccine drug products that contain on the order of ˜200 μg/mL of an mRNA construct. While the data in 1004 do not indicate an upper limit of quantification because the data are still linear at the highest concentrations tested, samples can always be diluted down into the appropriate working range prior to analysis, so this does not represent a limitation.

### Example 9

### Detection of mRNA in Coding Region (FIGS. 11-14)

FIG. 11 is a schematic of an embodiment that facilitates the specific capture of an mRNA construct by capturing a portion of the coding region (open reading frame) and labeling the 5′ cap. A polynucleotide capture agent is printed in a microarray spot 1101 comprising a synthetic DNA nucleotide with a sequence 1102 that is complementary to a portion of the coding region 1103 of an mRNA construct 1104. The mRNA construct 1104 has a 5′ cap 1105 that can be labeled using a label agent comprised of a fluorescently-conjugated anti-5′ cap antibody 1106. As long as the sequence of the capture agent is designed appropriately, such that it is specific for the mRNA target of interest, this detection scheme enables identity testing of the mRNA construct 1104 and enables concentration determination of the mRNA construct 1104 in either a monovalent sample (containing only a single mRNA construct) or a multivalent mixture (containing multiple unique mRNA constructs). An additional benefit of this approach is that the 5′ cap structure 1105 is most often identical between the multiple mRNA constructs in a multivalent mRNA-based vaccine or therapeutic, and an anti-5′ cap antibody 1106 therefore acts as a universal label for any mRNA construct containing the same 5′ cap structure.

Given that mRNA will only be detected when it is both captured and subsequently labeled, this detection scheme facilitates measurement of only the amount of mRNA construct that contains both the capture and label regions and may not be representative of total intact mRNA. This is because if there is a sequence truncation or other degradation of the mRNA in an area of the mRNA construct sequence that lies further towards the 3′ end of the mRNA construct (that is, outside of the contiguous sequence region between the capture and label), loss of this portion of the mRNA construct would not be detected.

One of ordinary skill in the art will realize that while an anti-5′ cap antibody labeling detection scheme may be utilized as shown in FIG. 11, this same effect may also be achieved utilizing the detection scheme shown in FIG. 12. FIG. 12 shows the same schematic described in FIG. 11, except that instead of an anti-5′ cap antibody label 1106, the detection label is a fluorescently-conjugated polynucleotide sequence 1201 designed to be complementary to the sequence region immediately adjacent to the 5′ cap 1202 on the mRNA construct. In most mRNA constructs relevant to mRNA-based vaccines and therapeutics, this region immediately adjacent to the 5′ cap structure will be an untranslated region (UTR). In a multivalent mRNA vaccine or therapeutic, this UTR is most often identical between the different mRNA constructs and would therefore act as a universal label for any mRNA construct containing the same UTR sequence.

One of ordinary skill in the art will also realize that while the detection schemes in FIG. 11 and FIG. 12 may be utilized, the same effect may be achieved utilizing the detection scheme shown in FIG. 13. FIG. 13 shows the same capture event as in FIG. 11 and FIG. 12, but the detection label is alternatively a fluorescently-conjugated poly-T polynucleotide sequence designed to be complementary to the poly A tail portion 1305 of the target mRNA construct 1304. In most mRNA constructs relevant to mRNA-based vaccines and therapeutics, there is a 3′ poly A tail present that is therefore complementary to a polyT detection label that can therefore act as a universal label for any mRNA construct containing a 3′ poly A tail.

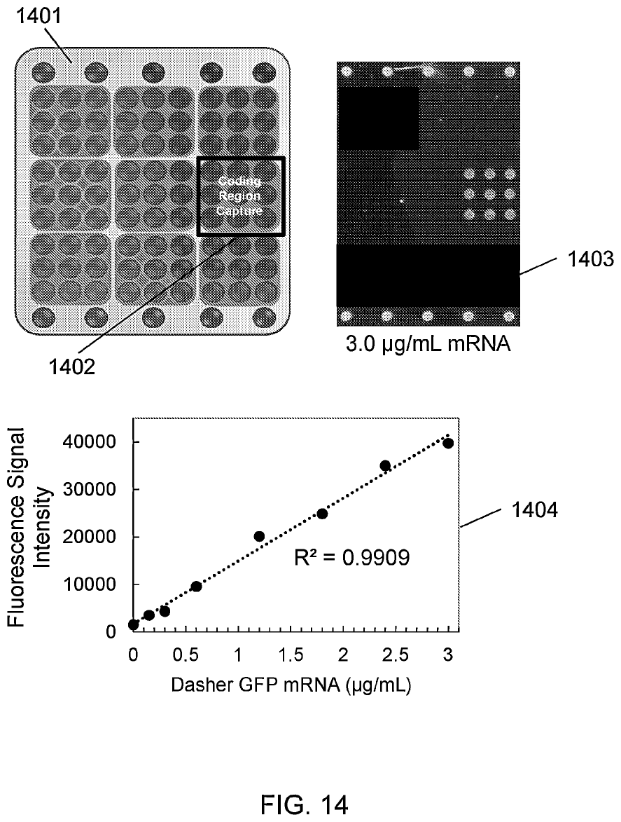

In FIG. 14, the microarray 1401 is comprised of 9 nucleic acid capture agents, each printed in 9 replicate spots, including a coding region capture agent 1402 of 20 nt in length with a 5′-amino-C6 modification to enable efficient coupling to the epoxide-functionalized glass microarray surface. The microarray 1401 is printed in 16 replicates onto a single epoxide-functionalized glass slide, and a 16-well silicone gasket is then adhered to create an individual ˜50 μL reaction volume for each microarray.

An mRNA construct including the open reading frame (ORF) or coding sequence for green fluorescent protein (GFP) was utilized. This mRNA construct does not include an untranslated region (UTR) but does include a ˜300-nt polyA tail similar to a typical mRNA construct. An initial 1:10 dilution of the stock mRNA was prepared in nuclease-free water, and then further diluted using a 2× binding buffer consisting of 8× saline sodium citrate (SSC), 4× Denhardt's reagent, and 0.2% sodium dodecyl sulfate (SDS), with subsequent serial dilutions made in lx binding buffer (all samples at final lx binding buffer concentration). Each dilution was applied to a separate replicate microarray 1401 for 60 minutes on a small rotation diameter orbital shaker to facilitate hybridization between the mRNA target and the capture agents on the microarray. The mRNA target-containing solutions were then removed, and the slides were washed with a wash solution containing 2× SSC and 0.1% SDS for 1 minute on the same orbital shaker. A fluorescently conjugated anti-5′ cap detection label antibody at 10 ug/mL in final buffer concentration of lx binding buffer was then added to all microarrays under analysis and allowed to incubate on the orbital shaker for 30 minutes. After detection label incubation, the slides were washed with first with a solution of 2× SSC and 0.1% SDS, followed by two serial washes with 0.2× SSC. The slides were then imaged with the VaxArray Imaging System (InDevR, Boulder, CO), and the fluorescence intensities on the coding region capture agent for the serial dilution subsequently analyzed. Representative fluorescence image 1403 shows an example image of a solution containing 3.0 μg/mL of mRNA hybridized to the coding region capture agent 1402 and subsequently labeled with an anti-5′ cap antibody.

Plot 1404 shows the linear fluorescence response obtained on the microarray for a serial dilution of the GFP mRNA construct, with a linear regression through the 7 dilutions producing a correlation coefficient (R²) of 0.99 on the coding region capture agent. Importantly, these data indicate successful capture of mRNA in the coding region. Because the coding region sequence is unique for each mRNA construct in a multivalent mRNA vaccine, this coding region capture ability is critical for application to multivalent mRNA vaccines such as those for influenza virus or other combinations of respiratory viruses. In addition, the linearity of response indicates that the amount of specific mRNA can not only be detected but can be quantified. Quantification of a sample containing an unknown amount of an mRNA would be quantified against an appropriate full-length, intact standard.

### Example 10

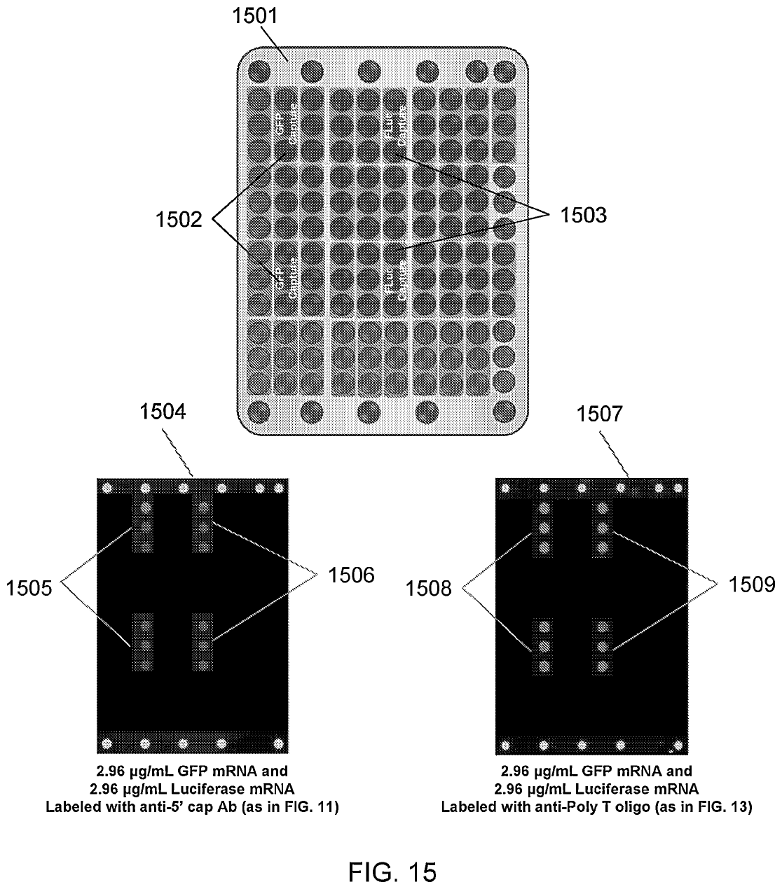

### Specificity and Linearity in a Bivalent mRNA Sample (FIG. 15-17)

Two different, commercially-available target mRNA constructs were mixed to mimic a multivalent vaccine. The first mRNA construct utilized has the open reading frame (ORF) or coding sequence for green fluorescent protein (GFP). This GFP mRNA construct does not include an untranslated region (UTR) but does include both a 5′ cap and a ˜300 nt 3′ polyA tail similar to a typical mRNA construct in a vaccine or therapeutic. The second mRNA construct includes the open reading frame (ORF) or coding sequence for firefly luciferase protein (FLuc). The FLuc mRNA construct does not include an untranslated region (UTR) but does include both a 5′ cap and a 120-nt 3′ Poly A tail similar to a typical mRNA construct in a vaccine or therapeutic.

In FIG. 15, the microarray 1501 is comprised of 20 nucleic acid capture agents, each printed in 6 replicate spots per capture throughout the array in two rows of 3 spots each. All capture agents have a 5′-amino-C6 modification to enable efficient coupling to the epoxide-functionalized glass microarray surface. Capture agent 1502 is a 20 nt length capture agent designed to be complementary to the GFP target mRNA construct. Capture agent 1503 is a 20 nt length capture agent designed to be complementary to the luciferase (FLuc) target mRNA construct. The microarray 1501 is printed in 16 replicates onto a single epoxide-functionalized glass slide, and a 16-well silicone gasket is then adhered to create an individual ˜50 μL reaction volume for each microarray.

An initial 1:10 dilution of stock GFP mRNA and stock FLuc mRNA were separately prepared in nuclease-free water. Three serial dilutions were analyzed: GFP mRNA only, FLuc mRNA only, and a mixture of GFP and FLuc mRNA with both mRNA constructs at the same concentration (GFP/FLuc). All three starting samples were further diluted using a 2× mRNA binding buffer consisting of 8× saline sodium citrate (SSC), 6% BSA, and 0.02% sodium azide, with subsequent dilutions in each series made in lx binding buffer (all samples at final 1× binding buffer concentration). Each dilution was applied to microarray 1501 for 60 minutes on a small rotation diameter orbital shaker to facilitate hybridization between the mRNA target and the polynucleotide capture agents on the microarray. The mRNA target-containing solutions were then removed, and the slides were washed with a wash solution containing 2× SSC and 0.1% SDS for 1 minute on the same orbital shaker. A fluorescently conjugated anti-5′ cap detection antibody at 5 μg/mL in final concentration or a fluorescently conjugated anti-5′ cap detection antibody at 5 μg/mL in final buffer concentration of lx mRNA binding buffer was then added to all microarrays under analysis and allowed to incubate on the orbital shaker for 30 minutes. After detection label incubation, the slides were washed with first with a solution of 2× SSC and 0.1% SDS, followed by two serial washes with 0.2× SSC. The slides were then imaged with the VaxArray® Imaging System (InDevR, Boulder, CO), and the fluorescence intensities generated on the coding region capture agent for each serial dilution subsequently analyzed.

Representative fluorescence images at the bottom of in FIG. 15 show examples of a bivalent GFP/FLuc containing sample. Representative fluorescence image 1504 shows capture of both mRNA targets in a bivalent GFP/FLuc sample using a detection label comprised of an anti-5′ cap antibody to which a fluorophore is conjugated to detect the 5′ end of the target mRNA, as depicted schematically in FIG. 11. Fluorescent spots 1505 show successful binding and subsequent detection labeling of the 2.96 μg/mL of GFP mRNA hybridized to the GFP coding region capture agent 1502. Fluorescent spots 1506 show successful binding and subsequent detection labeling of the 2.96 μg/mL of FLuc mRNA hybridized to the FLuc coding region capture agent 1503. Representative fluorescence image 1507 shows the capture of both mRNA targets in a bivalent GFP/FLuc sample labeled using a poly T polynucleotide detection label complementary to the 3′ poly A tail region of the mRNA target, as depicted schematically in FIG. 13. Fluorescent spots 1508 show successful binding and subsequent detection labeling of the 2.96 μg/mL of GFP mRNA hybridized to the GFP coding region capture agent 1502. Representative fluorescence spots 1509 successful binding and subsequent detection labeling of the 2.96 μg/mL of FLuc mRNA hybridized to the FLuc coding region capture agent 1503.

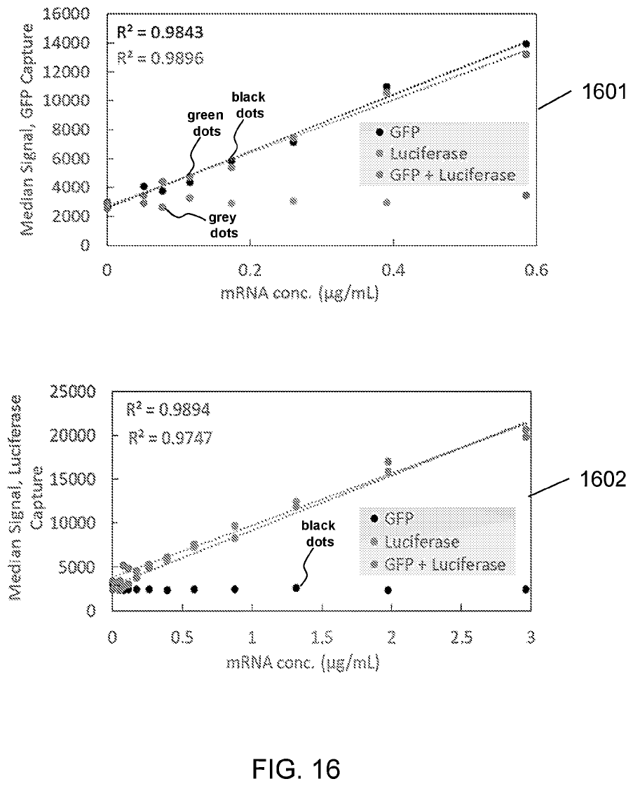

FIG. 16 shows the linear fluorescence response obtained on the microarray 1501 for a serial dilution of a monovalent GFP mRNA construct (black dots), monovalent FLuc mRNA construct (grey dots), and a bivalent GFP/FLuc mRNA mixture (green dots), subsequently labeled with an anti-5′cap antibody. The coding region sequence is unique for each mRNA construct in a multivalent mRNA vaccine, so these representative data indicate it is possible to design a microarray consistent with the current invention in which specific capture agents specifically bind to each unique mRNA construct to enable independent quantification in a multivalent mixture.

Plot 1601 shows the linear regression through the 7 dilutions of all three samples on a GFP coding region capture agent 1502. A flat response at signal equivalent to background (not dose-dependent) for a monovalent FLuc mRNA construct indicates that the GFP coding region capture agent 1502 shows specificity to the GFP mRNA construct. Importantly, both a monovalent GFP mRNA sample and a bivalent GFP/FLuc produce correlation coefficients (R²) of <0.98, and have similar response curves.

Plot 1602 shows the linear regression through the 7 dilutions of all three samples on a FLuc coding region capture agent 1503. A flat response at signal equivalent to background (not dose-dependent) indicates that the FLuc coding region capture agent 1503 shows specificity to the FLuc mRNA construct. Importantly, both a monovalent FLuc mRNA sample and a bivalent GFP/FLuC and produce correlation coefficients (R²) of <0.97, and have similar response curves.

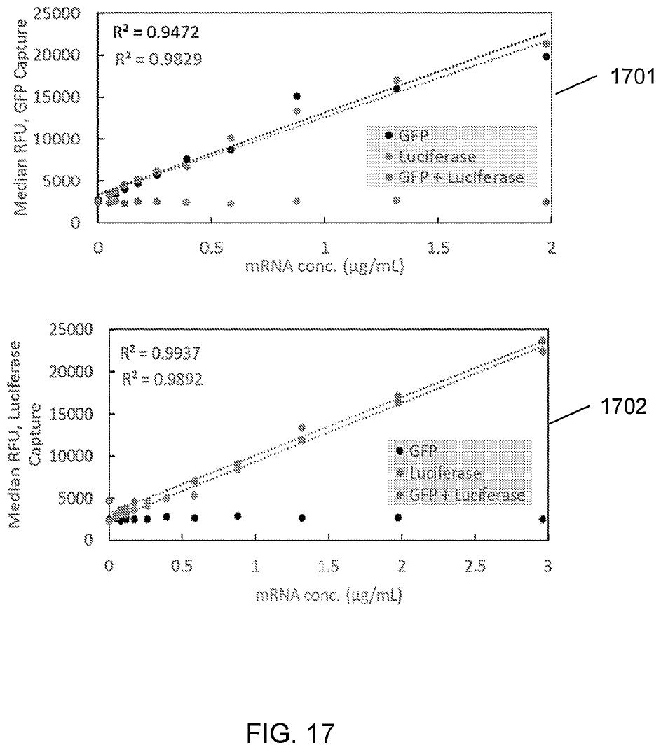

FIG. 17 shows the linear fluorescence microarray response obtained for a serial dilution of a monovalent GFP mRNA construct (black dots), monovalent FLuc mRNA construct (grey dots), and a bivalent GFP/FLuc mRNA mixture (green dots), subsequently labeled with a polyT polynucleotide label. The coding region sequence is unique for each mRNA construct in a multivalent mRNA vaccine, so these representative data indicate it is possible to design a microarray consistent with the current invention in which specific capture agents specifically bind to each unique mRNA construct to enable independent quantification in a multivalent mixture.

Plot 1701 shows the linear regression through the 7 dilutions of all three samples on a GFP coding region capture agent 1502. A flat response at signal equivalent to background (not dose-dependent) for a monovalent FLuc mRNA construct indicates that the capture agent 1502 shows specificity to the GFP mRNA construct. Importantly, both a monovalent GFP mRNA sample and a bivalent GFP/FLuC produce correlation coefficients (R²) of <0.94 and have similar response curves.

Plot 1702 shows the linear regression through the 7 dilutions of all three samples on a FLuc coding region capture agent 1503. A flat response at signal equivalent to background (not dose-dependent) for monovalent GFP mRNA construct indicates that the capture agent 1503 shows specificity to the FLuc mRNA construct. Importantly, both a monovalent FLuc mRNA sample and a bivalent GFP/FLuC produce correlation coefficients (R²) of <0.98 and have similar response curves.

Based on these data from FIG. 16 and FIG. 17, the specificity of the capture agents to their intended mRNA construct allows for simultaneous identification of all targets in a multivalent sample. The similar linearity of response curves between the monovalent and multivalent samples indicates that the provided systems and methods can be used in a multiplex fashion and used to characterize both monovalent and multivalent vaccines or therapeutics.

### Example 11

### Preparation of mRNA-Lipid Nanoparticle (LNP) Complexes

An mRNA construct including the open reading frame (ORF) or coding sequence for green fluorescent protein (GFP) was utilized. In one set of experiments, the four lipids present in the Pfizer-BioNTech COVID-19 Vaccine as described in the product insert were utilized: 4-hydroxybutyl)azanediyl)bis(hexane-6,1-diyl)bis(2-hexyldecanoate), 2[(polyethylene glycol)-2000]-N,N-ditetradecylacetamide, 1,2-distearoyl-sn-glycero-3-phosphocholine, and cholesterol. In this case, a lipid solution was prepared in ethanol at final concentrations of 0.062, 0.74, 0.052, 0.15 mg/mL in the 4 lipids listed above, respectively.

In another set of experiments, GenVoy-ILM ionizable lipid mix (Cat#NWW0056) included in the NanoAssemblr® Ignite™ Training Kit was purchased from Precision Nanosystems, Inc. This premixed lipid solution included a proprietary concentration ratio of the four lipids (1,2-dioleoyl-3-trimethylammonium-propane, 1,2-distearoyl-sn-glycero-3-phosphocholine, cholesterol, and PEG-DMG).

mRNA encapsulated in lipid nanoparticles (LNP) was prepared by combining the mRNA solution and the lipid-containing solution using the Ignite system from Precision Nanosystems, Inc. Briefly, ˜0.3 mg mRNA solution was combined with either a Precision Nanosystems lipids-containing solution or a Pfizer lipid-containing solution, and automatically combined using Precision Nanosystem's Ignite microfluidic mixing system using a flow rate ratio of 3 to 1 flow rate ratio for RNA (9 mL/min) to lipid-containing solution (3 mL/min) for a total flow rate of 12 mL/min. The output from the Ignite for both lipid systems was then analyzed downstream to determine the encapsulation efficiency using the RiboGreen assay as described below.

### Example 12

### Detection of Lipid Nanoparticle-Encapsulated mRNA

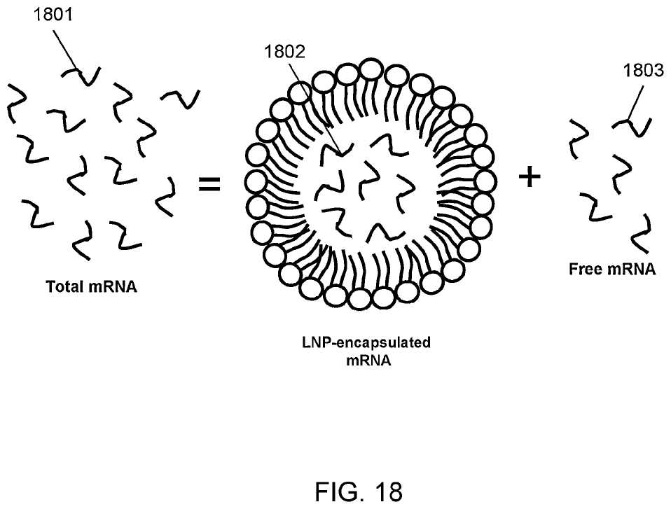

As schematically illustrated in FIG. 18, the total mRNA concentration 1801 in an mRNA-LNP formulation is the sum of the encapsulated mRNA 1802 plus the free mRNA 1803, as encapsulation efficiency is never 100%. In practice, the concentration of encapsulated mRNA 1802 can be measured by first measuring the concentration of total mRNA 1801 after the mRNA-LNP complex has been lysed with an appropriate detergent or other lysis reagent and subtracting the concentration of free mRNA 1803 (mRNA that is outside the LNPs) measured without a detergent or other lysis reagent to leave the mRNA-LNP intact. This is currently achieved via an assay such as the RiboGreen® assay. However, a major drawback of the RiboGreen® assay is the inability of applying this assay to a multivalent mRNA drug product because the RiboGreen® dye does not bind specifically to target mRNAs with different sequences and would therefore detect all mRNA constructs present non-specifically.

Quant-iT RiboGreen® RNA Assay Kit, (Invitrogen, cat#R11490) was used according to the manufacturer's instructions. Total mRNA in the mRNA-LNP complexes was measured via RiboGreen® assay by lysing the mRNA-LNP complexes in 10% Triton-X100 for 10 minutes at 37° C. and comparing absorbance measurements for the mRNA-LNP complex post lysis against a standard curve of the mRNA construct alone. As the RiboGreen assay will only measure mRNA that is outside of the LNP complexes, free mRNA was measured via RiboGreen® assay in the absence of a lysis step to leave the mRNA-LNP complexes intact. The amount of encapsulated mRNA is therefore measured by subtraction. Based on these RiboGreen® measurements, the encapsulation efficiency for the Pfizer mRNA-LNP was 89.7%, and 94.9% for the Precision Nanosystems mRNA-LNP.

As an alternative to the RiboGreen® assay and to demonstrate the utility of a microarray consistent with the current invention for the quantification of mRNA targets encapsulated within typical lipid nanoparticles present in mRNA vaccine samples, 3 replicates of encapsulated mRNA were prepared in triplicate in both the Pfizer LNP system and Precision Nanosystems LNP system as described above and determined to be at 3.3 μg/mL total mRNA for the Precision Nanosystems lipids mRNA-LNP samples, and 3.5 ug/mL for the Pfizer lipids mRNA-LNP samples. The replicate mRNA-LNP solutions were added to a hybridization solution containing 4× SSC, 2× Denhardt's solution, and 0.1% SDS and processed on a microarray consistent with that shown in FIG. 14 according to the microarray processing protocol described in Example 1. The capture agent on which the mRNA was quantified was in the coding region (as depicted by 1102 in FIG. 11), and the detection label utilized for this analysis was a fluorescent poly T polynucleotide that is complementary to the poly A tail of the GFP mRNA construct utilized (as depicted by 1301 in FIG. 13).

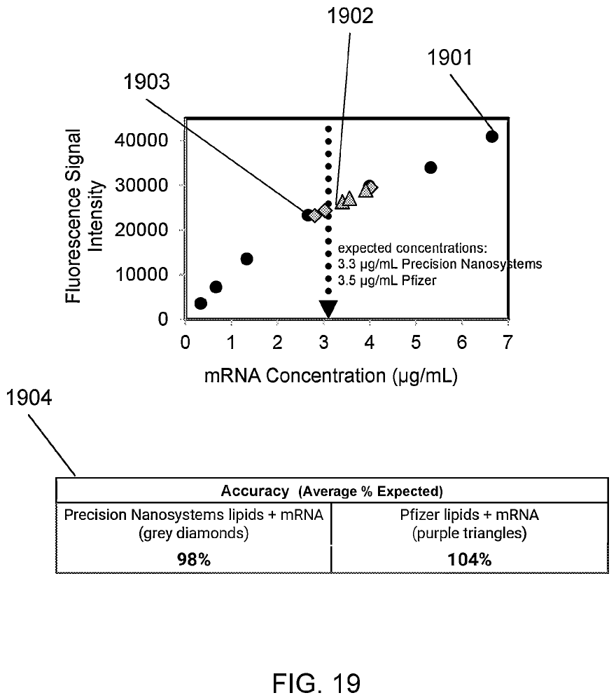

Shown in FIG. 19 are the microarray signals generated on the 640-659 nt coding region capture agent for these samples analyzed in triplicate alongside a standard curve 1901 of naked GFP mRNA of known concentration ranging from 0.33 to 6.66 μg/mL, with the Pfizer mRNA-LNP samples shown as grey triangles 1902 and the Precision Nanosystems mRNA-LNP samples shown as grey diamonds 1903. The standard curve was fit with a moving 4-point linear fit in which subsequent 4-point regions of the standard curve are each fit to a linear regression. The equation of the fit for each 4-point region meeting minimum linearity requirements and spanning the signal range in question was used to back-calculate the concentration of mRNA in the unknown sample, and the average of the back-calculated concentrations reported for each sample. With an expected concentration of mRNA of 3.3 ug/mL for the Precision Nanosystems lipids mRNA-LNP and 3.5 ug/mL for the Pfizer lipids mRNA-LNP, the high accuracy of the microarray quantification is shown in table 1904. The microarray analysis of the Precision Nanosystems mRNA-LNP samples resulted in average accuracy over the 3 replicates of 98%, and the Pfizer mRNA-LNP samples resulted in average accuracy over the 3 replicates of 104%. These data indicate that an accurate measurement of encapsulated mRNA can be achieved using the microarray method described herein.

Importantly, the data in FIG. 19 were generated without performing any extraction of the mRNA to remove the lipid molecules from the mRNA prior to adding the samples to the microarray. Unlike current methods such as PCR-based methods or sequencing methods that require a complex upfront extraction procedure to separate and purify the mRNA away from the lipid molecules due to potential for assay inhibition, no complicated lipid extraction is needed prior to the microarray analysis described herein. A simple lysis of the samples in an appropriate buffer prior to applying to the microarray is all that is required to achieve accurate quantitation of LNP-encapsulated mRNA. Accordingly, any of the methods and systems provided herein may be a simple lysis system, without any complex extraction and/or purifying steps, so that the only substantive processing step is a lysis step.

### Example 13

### Microarray Detection of Bivalent Influenza mRNA Constructs

Influenza NA and HA mRNA construct coding region sequences from Freyn et al. Molecular Therapy, 2020, 28(7), 1569-1600 were synthesized commercially by Trilink Biotechnologies (San Diego, CA), including a ˜128 nt poly A tail and untranslated (UTR) regions at both the 3′ and 5′ ends. The NA construct codes for a full-length membrane-bound NA from pdm (post-2009) H1N1 A/Michigan/45/2015, and the shorter HA construct codes for the conserved HA stalk domain of pre-2009 H1N1 A/Brisbane/59/2007. G. 20 (top panel) shows a schematic illustration of the constructs.

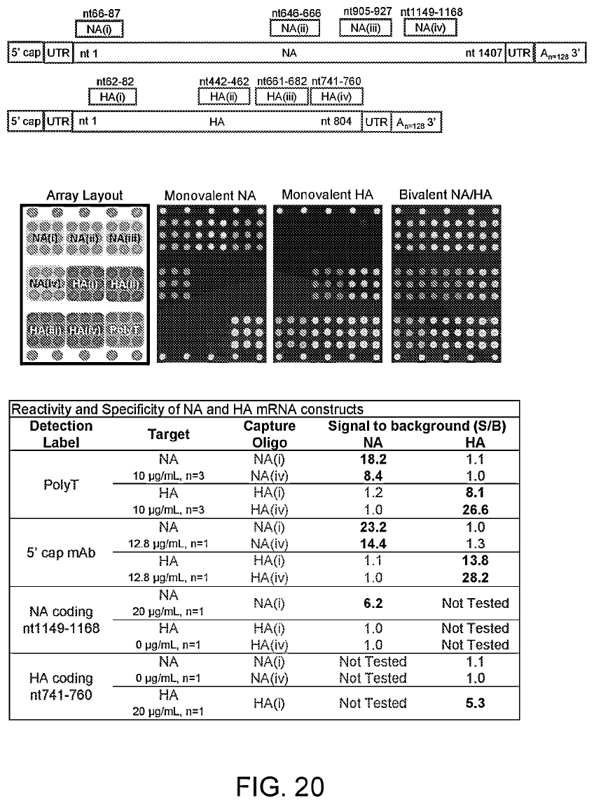

Polynucleotide capture agents (˜20-mers) are designed to target either the NA mRNA construct (21 captures) or HA mRNA construct (16 captures) based on the construct sequences. Unique sequence regions for the NA and HA mRNAs anticipated to provide specificity for each are identified by first aligning the mRNA sequences in BioEdit (v7.2., (Manchester, United Kingdom). The series of 19-24-nt length sequences identified that are unique to each construct are then imported into OligoAnalyzer (IDT; Coralville, IA) along with the HA and NA mRNA sequences to assess sequence parameters for anticipated microarray suitability, including: self-interactions with ΔG>−7.5 kcal/mole to minimize potential for hairpin formation, absence of low complexity sequence regions (such as repeat bases of >4 nt in length), and melting temperature >52° C. to enable room temperature hybridization. The ΔG is evaluated for the off-target construct to reduce potential for cross-annealing/non-specificity. Final specificity and subsequent down-selection are determined experimentally, with capture agents meeting appropriate performance requirements shown alongside the construct sequences in the top panel of FIG. 20. Polynucleotide capture agents are ordered at HPLC purification grade and with 5′ amino-C6 modification to enable covalent attachment to the microarray slides (IDT; Coralville, IA). In addition, a 30-nt polyT polynucleotide is designed as a capture agent to target the 3′ poly A tail of the mRNA constructs, also including a 5′-amino C6 modification. Select polynucleotides designed are utilized as detection labels and are synthesized by IDT with a 5′ Cy3 modification at HPLC purification grade to enable downstream fluorescence imaging. Oligonucleotides utilized as labels included the polyT oligonucleotide, NA nt1149-1168 label, and HA nt741-760 label. In addition, an anti-5′ cap antibody (MBL International Corp., Woburn, MA) is fluorescently conjugated in-house with an amine-reactive fluorescent dye antibody conjugation kit (Biotium, Fremont, CA) and used as a detection label.

Polynucleotide capture agents are printed onto epoxide-functionalized glass using a piezoelectric microarray printing system. An initial microarray with all 38 designed capture agents is printed and assessed for basic reactivity and specificity for the target construct using the polyT polynucleotide detection label. From this initial assessment, 4 capture agents for each construct from a range of positions along the coding region that exhibit both high reactivity and specificity are chosen for the final microarray design and printed. The microarray layout and associated representative images of monovalent HA, monovalent NA, and bivalent HA plus NA mRNA constructs hybridized to the microarray are shown in the center panel of FIG. 20.

To conduct the assay, slides are first equilibrated at 25° C. for 30 minutes and placed inside a humidity chamber (VX-6204, InDevR Inc.). Microarray slides are pre-washed with 50 μL 1× mRNA Wash Buffer 1 (VXI-6317, InDevR Inc.) in a humidity chamber on a shaker at 80 rpm for 1 minute at 25° C. All subsequent washes and incubations are on a shaker at 80 rpm at 25° C. After the wash, samples are diluted in an optimized mRNA Oligo Binding Buffer (VXI-6316, InDevR Inc.) at a final lx concentration and applied to designated arrays. Slide(s) are incubated for 1 hour and then washed with mRNA Wash Buffer 1 for 1 minute, followed by detection label incubation for 30 minutes. The slide(s) are then washed with mRNA Wash Buffer 1 once, and mRNA Wash Buffer 2 (VXI-6318, InDevR Inc.) twice prior to drying the slides by first pipetting off excess liquid from the wells followed by centrifugation for 10 s to remove any remaining liquid. Slide(s) are then imaged, and data analyzed using the VaxArray® Imaging System (VX-6000, InDevR Inc.).

Specificity and Reactivity. Specificity of the capture agents for the respective HA or NA target mRNA is verified using monovalent naked mRNA at 10 μg/mL. A label-only blank (no mRNA) is also analyzed to evaluate any direct binding of the detection labels to the capture oligos. Signal to background (S/B) ratios on all capture oligos on the microarray are calculated to assess reactivity and specificity, with a minimum reactivity threshold defined as a signal to background for the target mRNA of at least 3.0, and ideal specificity defined as a signal to background of 1.0 on off-target capture agents.

The middle panel of FIG. 20 shows a microarray layout alongside representative fluorescence images of monovalent NA, HA, and bivalent NA/HA, all analyzed at 10 μg/mL mRNA and labeled with the polyT polynucleotide detection label. These images qualitatively indicate that the microarray capture agents targeting designed for each mRNA construct generate specific signal for the intended mRNA target, that the specific signals vary for each of the capture agents implying different binding affinities, and that the off-target capture agents do not produce signal above background. These data using the polyT polynucleotide detection label are shown quantitatively in the highlighted rows in the table at the bottom panel of FIG. 20 in which the monovalent samples generated signal to background ratios (S/B) on the capture agents ranging from 8.1 to 26.6 indicating strong reactivity. In contrast, off-target capture agents resulted in S/B≤1.2, indicating no appreciable non-specific signal.

Reactivity and specificity for alternative detection schemes including coding region capture agent combined with anti-5′ cap detection label and coding region capture agent combined with coding region detection label are also investigated, as highlighted in the table at the bottom panel of FIG. 20. In all cases, the target mRNA generated S/B values ranging from 5.3 to 28.2, indicating good reactivity, whereas all off-target capture agent S/B ratios are ≤1.3. These data indicate the independent detection of multiple mRNAs in a multivalent mixture for a variety of assay detection principles, and the absence of signal for an off-target mRNA, both important for use as an identity test.

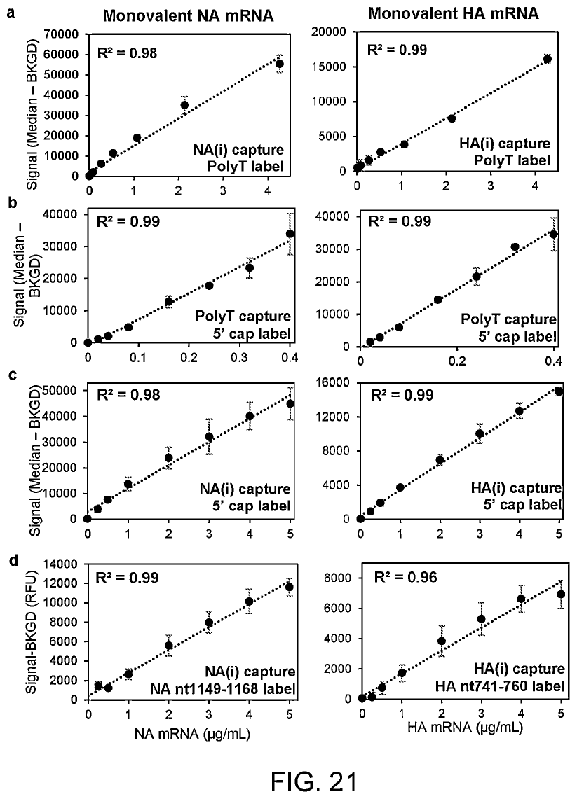

FIG. 21 highlights the average response for triplicate 8-point response curves on the NA(i), HA(i), and polyT captures for monovalent NA (left column) and HA (right column) mRNA using four different capture agent and detection labeling strategies. All detection schemes represented (as noted in each panel in FIG. 21) demonstrate good linearity with dilution, with all R²>0.96 for a single linear fit through each dataset. The data in FIG. 20 indicating microarray specificity in a bivalent mixture combined with the data in FIG. 21a demonstrating linearity with response for specific capture and universal labeling via the polyA tail in a bivalent mRNA mixture over a vaccine-relevant concentration range are important proof points for application to mRNA quantification in multivalent vaccine samples for bioprocess development and optimization. FIG. 21b highlights response curves for capture via the 3′ polyA tail at one end and labeling via the 5′ cap using an anti-5′ cap detection label antibody at the opposite end of the mRNA construct. This detection scheme can add value for monovalent formulations by enabling quantification of full-length mRNA as a measure of mRNA integrity. FIG. 21c highlights linearity of response for capture in the coding region for each construct and subsequent universal detection labeling of the 5′ cap using an anti-5′ cap detection label antibody. FIG. 21d demonstrates the ability for the assay to capture in the coding region and be labeled in the coding region for added specificity given that the polynucleotide detection labels are also specific to each construct.

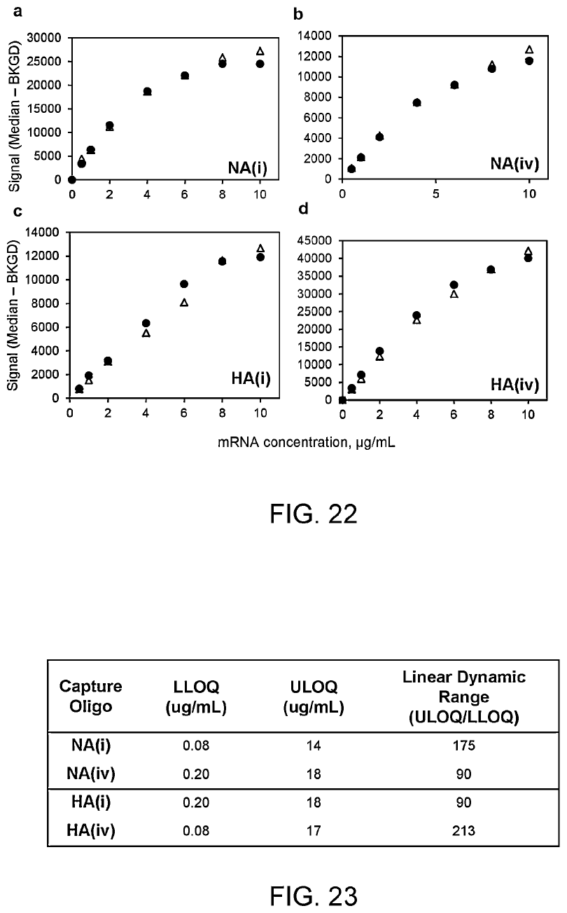

FIG. 22 shows a comparison of signal responses for monovalent mRNAs (closed circles) as well as a bivalent mixture of NA/HA mRNA (open triangles) analyzed on the VaxArray Assay at equal concentrations of each mRNA, all labeled with the polyT polynucleotide detection label. As shown in FIG. 22a and FIG. 22b, the response curves for the NA mRNA component in a monovalent vs. bivalent preparation in which HA mRNA was also present are essentially equivalent. Likewise, in FIG. 22c and FIG. 22d, the response curves for the HA mRNA component are also quite similar regardless of whether alone or in a bivalent mixture. Importantly, these data show no interference from the off-target mRNA in the presence of the target mRNA and indicate independent quantification is feasible in a bivalent mixture. Given the similarity of responses, the limits of quantification and accuracy/precision assessments are conducted using on bivalent HA/NA mixtures.

Limits of Quantification. To estimate lower and upper limits of quantification (LLOQ and ULOQ), monovalent HA and NA mRNA constructs are combined to formulate a bivalent stock sample. From the bivalent stock, 3 replicate 16-point dilution series are prepared individually and processed by the assay as described. The average of the 3 replicates is determined at each concentration, and a moving 4-point linear fit is applied to each dataset, calculating the slope and R² for each regression. The LLOQ is estimated by determining the concentration (within the lowest 4-point concentration range meeting minimum R²>0.95 requirement) at which the signal is equal to the average blank signal+5σ (where σ=standard deviation of the background signal). To verify the LLOQ, a bivalent 8-point dilution series that spanned the approximate LLOQ for each capture is created as a standard curve and analyzed alongside 8 different concentrations (n=4 at each concentration) of the bivalent sample near the approximate LLOQ. The same 4-point moving fit described above is applied to the standard curve, and the concentrations in each replicate sample back calculated. The verified LLOQ is reported as the lowest concentration at which both the % difference from expected (accuracy) and the % relative standard deviation (RSD) (precision) of the 4replicates are both less than 15%. ULOQ is approximated by determining the concentration at which the signal is 90% of the maximum observed signal within the previously determined concentration range. Similar to the LLOQ determination, the ULOQ is verified by evaluating 8 concentrations (n=4 each concentration) near the approximated ULOQ and determining the highest concentration at which the precision and accuracy requirements are met. Dynamic range of the assay is calculated as ULOQ/LLOQ.

FIG. 23 shows the values obtained, with LLOQ ranging from 0.08 μg/mL to 0.2 μg/mL, and ULOQ ranging from 14 μg/mL to 18 μg/mL. The dynamic range (ULOQ/LLOQ) for all four capture agents is >90×. Given mRNA construct concentrations of 200 μg/mL in on market COVID-19 mRNA vaccines, these data demonstrate an ability to quantify over a vaccine-relevant concentration range for application to formulated vaccine samples. For bioprocess samples (bulk mRNAs) that are likely at significantly higher concentration, a larger upfront dilution can readily bring samples into the appropriate working range.

Accuracy and Precision. Three users analyze eight (8) replicates of a contrived bivalent HA/NA mRNA sample at 2.4 μg/mL (3 users×8 replicates=24 replicates) using the polyT oligo detection label at 1 μM alongside an 8-point standard curve of the same contrived bivalent HA/NA sample. The accuracy to be investigated, therefore, is the accuracy of a “check standard”, and not the accuracy compared to an alternative standard method. To investigate accuracy, the concentration in each replicate is determined by back-calculating against the fit to the average of the 3 standard curves as the expected value. Accuracy is calculated as the % of expected (measured divided by expected, expressed as a percentage), and quantified for each user individually and over the three users combined. Assay precision is measured for each user as well as over all three users combined and expressed as % RSD of replicate measurements.

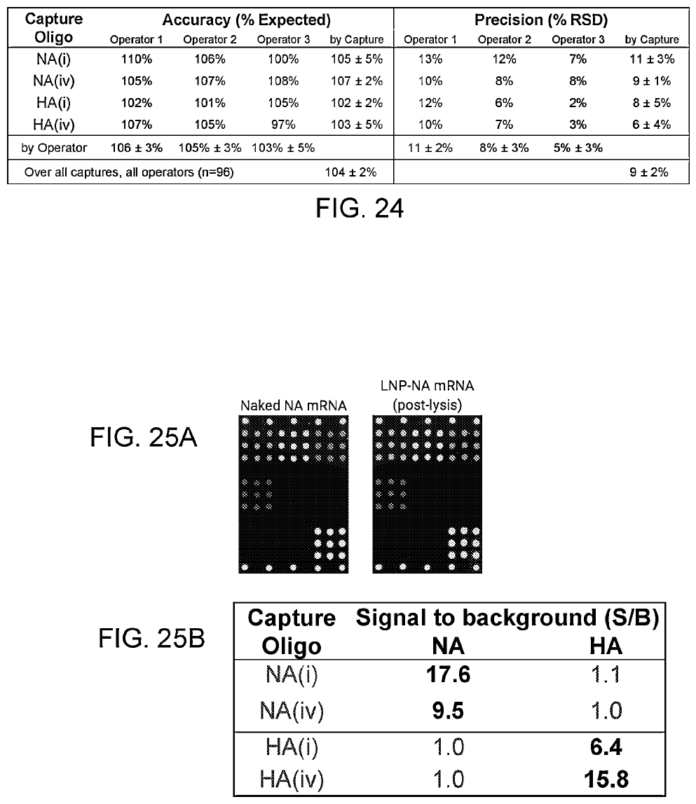

FIG. 24 shows accuracy and precision for each individual operator for the 2 capture agents for each target, combined over all three operators for each capture agent, and combined over all operators and all capture agents. Single operator accuracy ranged from 97% to 110% over the 4 capture agents, with an overall average of 104 (±2) %. Single operator precision ranged from 3% to 13% RSD over the 4 capture agents, with an overall average of 9 (±2) %. No statistically significant differences is observed between operators over all capture agents shown, as demonstrated by a two-tailed t-test executed using Microsoft Excel (all p values were >0.14).

### Example 14

### Detection of Lipid Nanoparticle (LNP)-Encapsulated Influenza Constructs

LNP Encapsulation of mRNA. The Precision Nanosystems Inc. (Vancouver, BC, Canada) NanoAssemblr® Spark™ microfluidic mixer is used to encapsulate mRNA in LNPs. Following the manufacturer's protocol, NA and HA naked mRNA constructs are encapsulated at known quantity in the manufacturer's provided LNP mix containing a proprietary mix of the following four lipids in ethanol: 1,2-Dioleoyl-3-trimethylammonium propane (DOTAP), distearoylphosphatidylcholine (DSPC), cholesterol, and 1,2-Dimyristoyl-rac-glycero-3-methoxypolyethylene glycol-2000 (PEG-DMG). Encapsulated materials were stored at +2-8° C. prior to analysis.

Quantity of encapsulated mRNA and encapsulation efficiency are measured with the Quant-it™ RiboGreen RNA Assay Kit (cat#R11490; Invitrogen, Waltham, MA) following the manufacturer's protocol. Briefly, total mRNA was measured using the RiboGreen assay with an upfront lysis step in 2% Triton X-100 (SX100-500ML; Sigma Aldrich, St. Louis, MO) to lyse the LNPs. Free mRNA was measured in the absence of the lysis step, and encapsulated mRNA is measured via subtraction of free mRNA from total mRNA, with encapsulation efficiency expressed as encapsulated mRNA divided by total mRNA, expressed as a percentage. LNP-encapsulated NA and HA mRNAs contained ˜75-85 μg/mL total mRNA, depending on the experiment, with measured encapsulation efficiencies >90%. Measurement of encapsulated mRNA is performed within 48 hours of the subsequent VaxArray experiment to account for any change in encapsulation efficiency of the LNP/mRNA samples during storage.

To conduct the microarray assay on lipid nanoparticle-encapsulated constructs, slides are first equilibrated at 25° C. for 30 minutes and placed inside a humidity chamber (VX-6204, InDevR Inc.). Microarray slides are pre-washed with 50 μL 1× mRNA Wash Buffer 1 (VXI-6317, InDevR Inc.) in a humidity chamber on a shaker at 80 rpm for 1 minute at 25° C. All subsequent washes and incubations are on a shaker at 80 rpm at 25° C. After the wash, samples are lysed in 1% Triton X-100 at 37° C. for 10 minutes, and then subsequently diluted to the needed testing concentration in an optimized mRNA Oligo Binding Buffer (VXI-6316, InDevR Inc.) at a final 1× concentration, supplemented with 1% Triton X-100) and applied to designated arrays. Slide(s) are incubated for 1 hour and then washed with mRNA Wash Buffer 1 for 1 minute, followed by detection label incubation for 30 minutes. The slide(s) are then washed with mRNA Wash Buffer 1 once, and mRNA Wash Buffer 2 (VXI-6318, InDevR Inc.) twice prior to drying the slides by first pipetting off excess liquid from the wells followed by centrifugation for 10 s to remove any remaining liquid. Slide(s) are imaged on the VaxArray® Imaging System (VX-6000, InDevR Inc.), and downstream data analysis is performed using the VaxArray Analysis Software.

Specificity and Response Curves. For assessing general response in encapsulated materials, LNP-encapsulated monovalent NA and HA mRNA and a bivalent mixture of NA and HA mRNA are analyzed and compared to assess similarity of response. Blank LNPs (no mRNA encapsulated) are assessed by VaxArray post-lysis as described above, with no false positive results on the microarray observed as demonstrated by no S/B >1.0, indicating a lack of interference from the presence of the lipids. FIG. 25A (top panel) compares a representative fluorescence image of the same 2 μg/mL concentration of NA mRNA in naked (unencapsulated) and LNP-encapsulated samples (encapsulated sample is lysed as noted above prior to analysis is described), both using the polyT polynucleotide detection label and imaged at the same exposure time. These images indicate that the LNP-mRNA complex generates a similar specific response to the naked mRNA and does not produce off-target signal on the HA-specific capture agents. The quantitative analysis in FIG. 25B indicates that when LNP-encapsulated monovalent mRNA is labeled with the polyT polynucleotide detection label is analyzed, S/B on target captures are ≥6.4, and off-target polynucleotide capture agents results in S/B ≤1.1, indicating good reactivity and specificity. Importantly, these data demonstrate no requirement for upfront purification of the mRNA from LNPs, and no false positive interference from the lipid components used as a common delivery vehicle, enabling use of the assay for formulated vaccine samples.

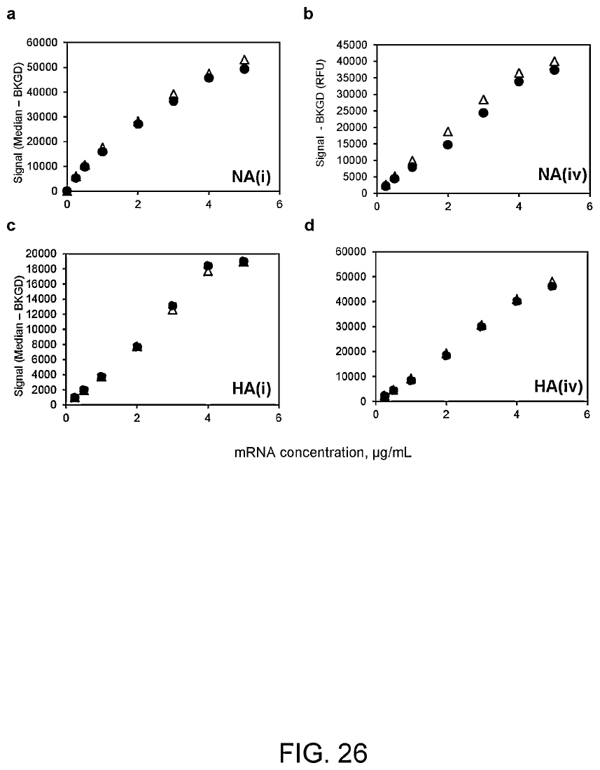

FIG. 26 shows a comparison of signal response curves for monovalent LNP-encapsulated mRNA as well as a bivalent mixture of LNP-encapsulated NA and HA mRNA at equal concentrations in each mRNA, all labeled with the polyT polynucleotide detection label. As shown in FIG. 26a and FIG. 26b for the two NA-specific capture oligos, the response curves for the NA mRNA component in monovalent vs. bivalent preparations in which HA mRNA is also present are quite similar. Likewise, in FIG. 26c and FIG. 26d, the response curves for the HA mRNA component are also similar for both the HA-specific capture agents, regardless of whether alone or in a bivalent mixture with NA mRNA. Importantly, these data show no interference from the off-target mRNA in the presence of the target mRNA and indicate independent quantification is feasible in a bivalent LNP-encapsulated mRNA mixture.

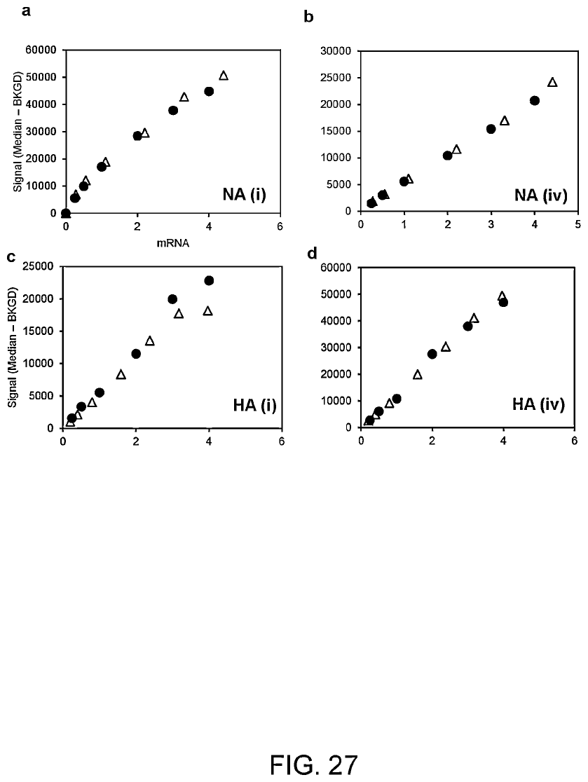

FIG. 27 shows a comparison of signal responses for bivalent LNP-encapsulated mRNA as well as bivalent naked mRNA (no LNPs), both processed with 1% Triton X-100 as described earlier to ensure matrix matching and using the polyT polynucleotide detection label. The response curves for both of the NA-specific capture agents in FIGS. 27a and 27b are very similar for both the naked and LNP-encapsulated samples, as are the response curves for both HA-specific capture agents in FIGS. 27c and 27d. Importantly, these data indicate that a naked mRNA standard should be appropriate for enabling accurate quantification in LNP-encapsulated samples over the assay linear range, provided both are in the same matrix.

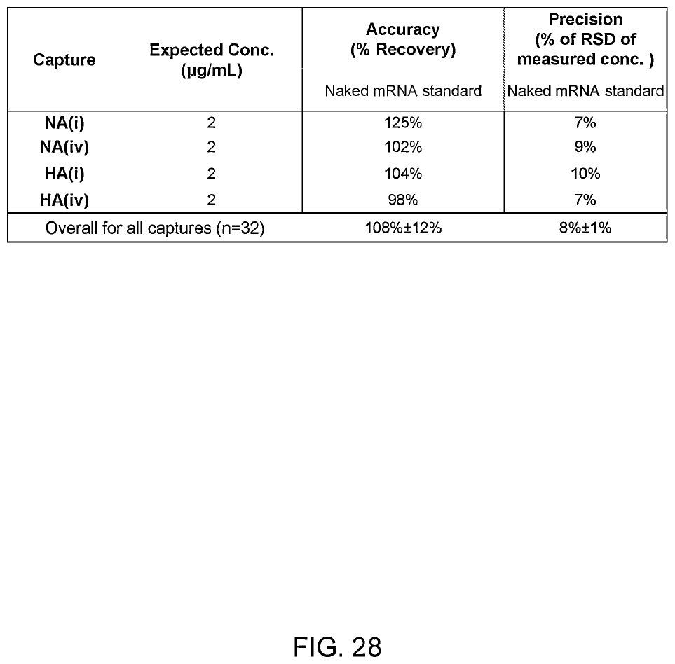

Accuracy and Precision. For accuracy and precision measurements, monovalent NA and HA mRNA were separately encapsulated in LNPs and analyzed post-lysis in 8 replicates alongside a standard curve of naked mRNA also pre-treated in 1% Triton X-100 to match the sample matrix. Replicates are prepared at 2 μg/mL total mRNA. Signals from the lysed replicates are back-calculated using the fit equation to the standard curve to determine the measured concentration and associated accuracy and precision. A moving 4-point linear fit is applied to each dataset, and R² for each regression was >0.95 except for the top point. FIG. 28 shows quantification accuracy and precision for these LNP-encapsulated monovalent mRNA samples for the 2 capture agents for each target mRNA. Accuracy for the NA(i) and NA(iv) capture agents are 125% and 102% of expected concentration, respectively, and HA(i) and HA(iv) capture agents produced 104% and 98% of expected, respectively. The average accuracy was 108 (±12) %. Precision of the back-calculated concentration, expressed as the % RSD of the 8 replicates, ranged from 7% to 10% with an overall average precision of 8 (±1) %. While the NA(i) capture agent showed slightly higher accuracy than expected, the accuracy and precision data overall were generally quite similar to data generated for the naked mRNA samples shown in FIG. 24.

We claim:

1. A method for characterizing a polynucleotide, the method comprising the steps of:
providing a capture agent on a substrate surface;
introducing a sample containing the polynucleotide to the capture agent;
binding at least a target region of the polynucleotide to the capture agent to form a polynucleotide and capture agent complex;
removing unbound material from the substrate surface;
labeling the polynucleotide and capture agent complex with a label to form labeled complexes; and
detecting the labeled complexes;
thereby characterizing the polynucleotide.

2. The method of claim 1 for multiplex characterization of a plurality of mRNAs.

3. The method of claim 1 or 2, wherein the characterizing comprises one or more of:
polynucleotide identity;
polynucleotide quantity;
polynucleotide integrity; and/or
polynucleotide stability.

4. The method of any claims 1-3, wherein the sample is from an mRNA vaccine.

5. The method of any of claims 1-4, wherein the polynucleotide comprises one or more of:
a native ribonucleotide;
a native nucleotide;
a non-native ribonucleotide; and/or
a non-native nucleotide.

6. The method of any of claims 1-5, comprising a plurality of unique capture agents on the substrate surface to provide multiplex characterization of a plurality of polynucleotides.

7. The method of claim 6, wherein the multiplex characterization is used to:
identify a plurality of mRNAs in a vaccine manufacture process;
identify a plurality of mRNAs in a therapeutic;
characterize a plurality of mRNAs in a vaccine; and/or
characterize a plurality of mRNAs in a therapeutic.

8. The method of any claims 1-7, wherein the capture agent comprises:
a target-specific nucleic acid capture sequence having sequence complementarity to at least a portion of the polynucleotide corresponding to a target polynucleotide sequence, wherein the target-specific nucleic acid capture sequence specifically hybridizes to the target polynucleotide sequence.

9. The method of claim 8, wherein the capture agent comprises a plurality of unique capture agents configured to specifically hybridize to a plurality of unique target polynucleotide sequences, wherein each individual unique capture agent specifically hybridizes to one unique target polynucleotide sequence, thereby providing multiplex detection.

10. The method of any of claims 1-9, wherein a plurality of capture agents are provided on the substrate surface in an array, and the detecting step comprises:
detecting a spatial pattern of complexes.

11. The method of any of claims 1-10, wherein the detecting step comprises:
assigning a threshold value relative to an optical signal generated by the labeled complexes; and
identifying a positive complex at locations on the substrate surface for the optical signal generated by the labeled complex that exceeds the threshold value.

12. The method of claim 11, further comprising the step of:
identifying a negative complex at locations on the substrate surface for an optical signal that is less than the threshold value; and
using the combination of positive complex locations and negative complex locations to thereby characterize the polynucleotide.

13. The method of any of claims 1-12, further comprising the steps of:
a) introducing a standardized polynucleotide having a known polynucleotide concentration to the substrate surface;
b) binding the standardized polynucleotide to the capture agent to form a standardized polynucleotide and capture agent complex;
c) removing unbound material from the substrate surface;
d) labeling the standardized polynucleotide and capture agent complex with the label to form labeled standard complexes;
e) detecting the labeled standard complexes;
f) repeating steps (a)-(e) for the standardized polynucleotide having a different known polynucleotide concentration, wherein the repeating steps (a)-(e) are for a repeat number that is greater than or equal to 2;
g) generating a calibration curve from each of the detecting steps for each of the at least three standardized polynucleotides having different known polynucleotide concentrations; and
h) using the calibration curve to quantify an amount of the polynucleotide in the sample.

14. The method of claim 13, wherein the capture agent comprises a plurality of unique capture agents provided in an array on the substrate surface, with each unique capture agent specific to a unique target polynucleotide sequence and the label is a universal label.

15. The method of claim 13, wherein the capture agent further comprises a universal capture agent configured to bind to a conserved region of a plurality of unique target polynucleotide sequences, and a plurality of labels, each label configured to bind to a unique complex; and

16. The method of claim 13, wherein the capture agent further comprises a plurality of capture agents and a plurality of labels, each of the plurality of capture agents configured to bind to the unique target polynucleotide and each of the plurality of labels configured to bind a unique target region of the polynucleotide sequences.

17. The method of any of claims 1-16, wherein the characterization comprises:
assessment of integrity or stability of the polynucleotide as a function of time and/or storage conditions.

18. The method of any claims 1-17, wherein the sample is an mRNA-containing vaccine or therapeutic against a virus of interest or a genetic disease.

19. The method of claim 18, wherein the virus of interest is a coronavirus, an influenza virus, and/or an emergent virus.

20. The method of claim 18, wherein the sample is an mRNA influenza vaccine, and the capture agent has sequence complementarity to a specific subtype or lineage of influenza virus.

21. The method of claim 20, wherein the capture agent comprises a plurality of unique capture agents, each capture agent having sequence complementarity to a different subtype or lineage of influenza virus.

22. The method of any of claims 20-21, wherein the detecting step further comprises determining an identity and/or quantity of the polynucleotide that is mRNA in the sample.

23. The method of any of claims 20-22, wherein the capture agent comprises:
a first capture agent configured to specifically hybridize to an mRNA corresponding to a conserved region of influenza hemagglutinin (HA) gene; and/or
a second capture agent configured to specifically hybridize to an mRNA corresponding to a conserved region of influenza neuraminidase (NA) gene;
wherein the conserved HA and NA regions are conserved amongst all of a targeted influenza subtype or lineage so that the method of characterizing the mRNA can characterize different seasonal influenza mRNA vaccines without updating the capture agents.

24. The method of any of claims 1-23, wherein the sample is a multivalent mRNA vaccine, including a quadrivalent mRNA influenza vaccine.

25. The method of any of claims 1-24, wherein the characterizing corresponds to confirming a presence of a portion of an mRNA construct sequence, including the portion of mRNA corresponding to: a 5′ cap, an untranslated region, a coding sequence, or a 3′ polyA tail.

26. The method of any claims 1-25, wherein the sample comprises a therapeutic having mRNA for a genetic disease treatment.

27. The method of any of claims 1-26, further comprising the step of:
linearizing a target polynucleotide sequence of the polynucleotide to facilitate target polynucleotide sequence binding to a respective capture agent by relaxing or eliminating polynucleotide secondary structure.

28. The method of claim 27, wherein the linearizing comprises introducing to the sample one or more of: heat, a chaperone, an additive and/or a detergent.

29. The method of any of claims 1-28, used in a quality control application.

30. The method of claim 29, wherein the quality control application is a multivalent mRNA vaccine, and one configuration of capture agents on the substrate surface is used for all quality control assessments related to singleplex characterization of mRNA vaccine individual polynucleotide constituents and multiplex characterization of all mRNA polynucleotides in the multivalent vaccine.

31. A microarray mRNA characterization system configured to perform any of the methods of claims 1-30 to characterize one or more mRNA polynucleotides.
